(19) United States
(12) Patent Application Publication
(10) Pub. No.: US 2025/0326790 A1
(43) Pub. Date: Oct. 23, 2025

(54) PEPTIDES BINDING TO HYPOXIA INDUCIBLE FACTORS AND THEIR USE

(71) Applicant: UNIVERSITY OF SOUTHAMPTON, Highfield, Southampton Hampshire (GB)

(72) Inventor: Ali TAVASSOLI, Southampton Hampshire (GB)

(73) Assignee: UNIVERSITY OF SOUTHAMPTON, Highfield, Southampton Hampshire (GB)

(21) Appl. No.: 18/547,622

(22) PCT Filed: Feb. 25, 2022
(86) PCT No.: PCT/GB2022/050523

§ 371 Date: Aug. 23, 2023

(30) Foreign Application Priority Data
Feb. 25, 2021 (GB) 2102709.9

(51) Int. Cl. C07K 5/083; A61K 38/00; A61P 35/00

(52) U.S. Cl. CPC C07K 5/081; A61P 35/00; A61K 38/00

(57) ABSTRACT

There are a series of tripeptides according to the compound of formula (I) that inhibit the interaction of both HIF-1α and HIF-2α with Hlf-1 β by binding to the PAS-B domain of the α subunit of HIF. The tripeptides and methods disclosed herein are useful in treating diseases or conditions that involve the response to hypoxia.

### CROSS-REFERENCE TO RELATED APPLICATIONS

The present application is a national stage application claiming priority from PCT Application No. PCT/GB2022/050523, filed Feb. 25, 2022, which claims priority from GB Patent Application No. 2102709.9, filed Feb. 25, 2021, the disclosures of which are incorporated herein in their entireties.

### FIELD OF THE DISCLOSURE

The disclosure relates to therapeutic agents and methods for treating diseases or conditions that involve response to hypoxia.

### DESCRIPTION OF THE RELATED ART

Hypoxia is a state of reduced oxygen concentration that can arise under normal conditions such as embryonic development, but also plays a key role in multiple pathological conditions, such as cardiac arrest, stroke and cancer.¹ Hypoxia has particular relevance in cancers as solid tumours contain hypoxic regions (pO₂≤2.5 mmHg)² that occur due to tumour cell growth exceeding the capacity of the surrounding vascular infrastructure. Hypoxia inducible factors (HIF) are heterodimeric transcription factors that assemble in hypoxia and reprogram gene expression to allow survival and growth of cells in a low oxygen microenvironment.³⁻⁵ The expression of several hundred genes has been directly linked to HIF-1 activation, and genomic analysis of HRE sequences estimates that HIF-1 mediates the expression of up to 1% of the genome.^{6,7} Whilst HIF activity impacts a diverse set of cellular pathways, the primary means by which hypoxic response is enacted is through the reprogramming of glucose metabolism, and the promotion of angiogenesis and proliferation. This response is believed to promote an aggressive phenotype and prolong tumour survival. As such, HIFs has long been proposed to be an attractive target for cancer therapy.

HIF is a heterodimeric transcription factor, which comprises of an oxygen-sensitive α subunit, and a constitutively-expressed β subunit (also known as the aryl hydrocarbon nuclear receptor translocator, ARNT). There are 3 isoforms of the HIF-α that bind to HIF-1β, with HIF-1α and HIF-2α being responsible for orchestrating hypoxia-response. The α-subunit of HIF is continually expressed but subject to post-translational modifications by oxygen-dependent proline hydroxylases (PHD). The hydroxylation of two prolines (P402 and P564 in HIF-1α) enables recognition by the von Hippel-Lindau protein and its associated E3 ligase complex, which triggers rapid ubiquitination and proteasomal degradation. Thus, HIF activity is acutely oxygen-sensitive, with HIF-1α having a half-life of less than 5 minutes in normoxia. HIF-1α is not degraded in hypoxia due to the absence of the molecular oxygen required for prolyl hydroxylation. The subsequent increase in HIF-α concentration causes it to translocate to the nucleus where it forms a dimeric complex with the constitutively expressed HIF-β to form the active HIF transcription factor. HIF binds to numerous hypoxia-response elements (HRE) present in the genome to reprogramme hypoxic cells to allow their survival and growth. The protein-protein interaction of the α and β subunit of HIF is a potential key point of therapeutic intervention, with recent successes in disrupting this interaction in HIF-2 translating into a potential cancer treatment programme.

Previous work in this area has largely focused on the specific targeting of one isoform over the other; this has largely been due to the presence of a small molecule cavity only present in the 2α PAS-B domain of HIF-2 that allows selective targeting of this isoform. HIF-1α is expressed ubiquitously, whereas HIF-2α and HIF-3α appear to be expressed in a more tissue-specific or environmentally conditional manner. Interestingly, HIF-1α and HIF-2α appear to have non-redundant roles that each produce distinct phenotypes due to their distinct target genes and in tissues where both isoforms are expressed, they have synergistic roles in promoting the hypoxic response.

The present disclosure seeks to provide inhibitors that are capable of targeting both isoforms—HIF-1α and HIF-2α.

### SUMMARY OF THE PRESENT DISCLOSURE

The present disclosure provides a series of tripeptides that inhibit the interaction of both HIF-1α and HIF-2α with HIf-1 β by binding to the PAS-B domain of the α subunit of HIF.

Accordingly, in a first aspect of the disclosure, there is provided a compound of formula (I):

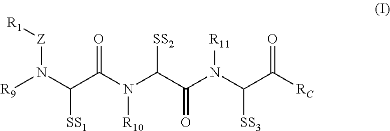

wherein Z═CO or SO₂ or H, wherein if Z is H there is no R₁;
R₉, R₁₀ and R₁₁ are independently H or methyl;
R₁=straight-chain or branched C₁-C₈ alkyl; CH-(3-6 membered ring)₂; (CH₂)₁₋₃O (CH₂)₀₋₃CH₃; or (CH₂)₀₋₃R₂;
R₂═NH(CH₂)₀₋₃CH₃; a 8-12 membered bicyclic ring optionally substituted by one or more of C₁-C₃ alkyl, HaI, CHHaI₂, CH₂HaI, and CHaI₃; or a 3-6 membered ring optionally substituted by one or more of C₁-C₃ alkyl, HaI, CHHaI₂, CH₂HaI, CHaI₃, CO(CH₂)₀₋₂CH₃, nitro group and (O)₀₋₁-3-6 membered ring optionally substituted by one or more of C₁-C₃ alkyl, HaI, CHHaI₂, CH₂HaI, and CHaI₃;
R_{C} is (i), (ii) or (iii):
wherein (i) is:

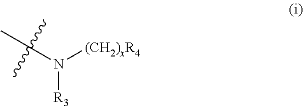

wherein R₃═H; or straight-chain or branched C₁-C₈ alkyl;
X=an integer of from 0 to 8;
R₄═H; CHHaI₂; CH₂HaI; CHaI₃; HaI; CCH; NR₅R₆; CH(COOMe)(CH₂)₁₋₄NHC(NH₂)(NH); or 3-6 membered ring optionally substituted by one or more of C₁-C₃ alkyl, HaI, NR₇R₈, and COCH₃;
R₅═H; or straight-chain or branched C₁-C₃ alkyl;
R₆═H; or straight-chain or branched C₁-C₃ alkyl; or 3-6 membered ring;
R₇═H; or straight-chain or branched C₁-C₃ alkyl;
R₈═H; or straight-chain or branched C₁-C₃ alkyl;
wherein (ii) is a 5-6 membered heterocycle comprising at least one nitrogen atom and optionally substituted by one or more of C₁-C₃ alkyl, HaI, CHHaI₂, CH₂HaI, CHaI₃, NR₇R₈, and COCH₃, wherein R_{c} is bonded via the at least one nitrogen atom;
wherein (iii) is OH;
wherein HaI is a halogen;
wherein SS₁, SS₂ and SS₃ are independently selected from one of the side-chains in list (a), list (b) or list (c):

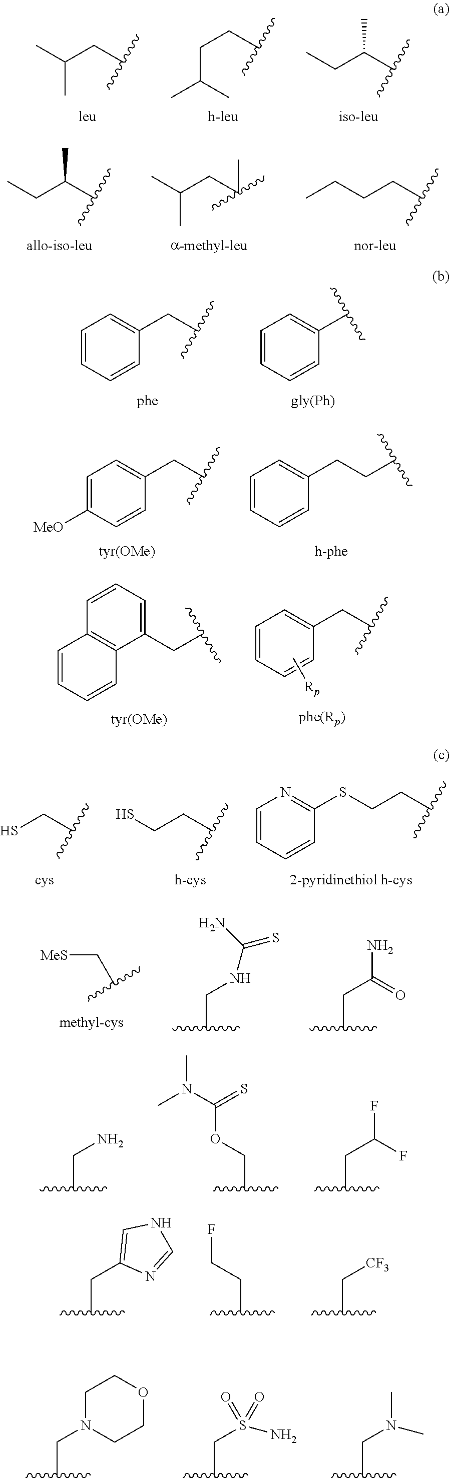

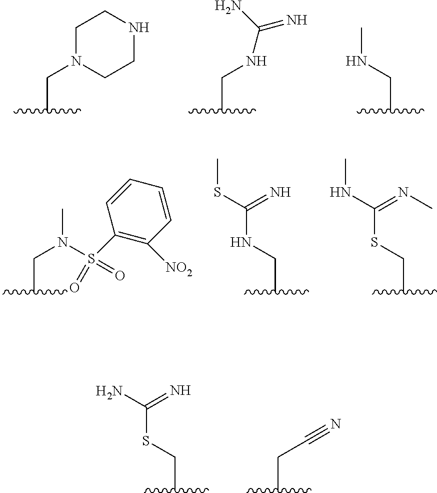

wherein SS₁, SS₂ and SS₃ are each selected from different lists;
wherein the phe(R_{P}) ring is substituted with one, two, or three R_{P} groups; and each R_{P} is independently I, Br, Cl, F, OH, Bz, NO₂, CN, or CF₃;
or a pharmaceutically acceptable salt thereof.

In a second aspect of the disclosure, there is provided a method of treating or preventing a disease or condition that involves a response to hypoxia by administering the compound of formula (I).

In a third aspect of the disclosure, there is provided a use of the compound of formula (I) as a medicament.

In a fourth aspect of the disclosure, there is provided a pharmaceutical composition comprising the compound of formula (I).

In a fifth aspect of the disclosure, there is provided a use of the compound of formula (I) in a method of manufacturing a medicament.

In a sixth aspect of the disclosure, there is provided a compound of formula (II), a compound of formula (V) and compound of formula (VII):

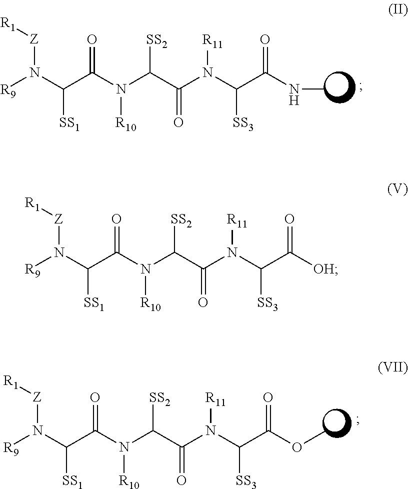

wherein Z, R₁, R₂, R₉, R₁₀, R₁₁, SS₁, SS₂ and SS₃ are as defined for the compound of formula (I);
or a protected derivative thereof or salt thereof.

In a seventh aspect of the disclosure, there is provided a compound of formula (IV):

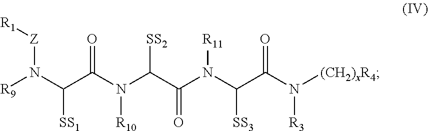

wherein Z, R₁, R₂, R₃, X, R₄, R₉, R₁₀, R₁₁, SS₁, SS₂ and SS₃ are as defined for the compound of formula (I);
wherein one or more of SS₁, SS₂ and SS₃ comprises a nucleophilic functional group and has a protecting group, such as StBu, on said functional group;
or a protected derivative thereof or salt thereof.

In an eighth aspect of the disclosure, there is provided a process for preparing a compound of formula (I), wherein R_{c} represents (i), R₃ represents H, X represents 0 and R₄ represents H, or a pharmaceutically acceptable salt thereof, which comprises removing the compound of formula (II) from a rink-amide resin and removing any protecting groups, wherein formula (II) is:

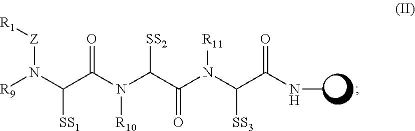

wherein Z, R₁, R₂, R₉, R₁₀, R₁₁, SS₁, SS₂ and SS₃ are as defined for the compound of formula (I).

In a ninth aspect of the disclosure, there is provided a process for preparing a compound of formula (II) or a protected derivative thereof or salt thereof, which comprises reacting a compound of formula (III):

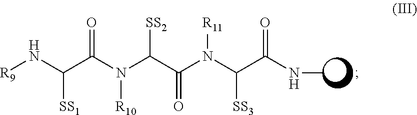

with a compound of R₁ZCl;
wherein Z, R₁, R₂, R₉, R₁₀, R₁₁, SS₁, SS₂ and SS₃ are as defined for the compound of formula (I).

In a tenth aspect of the disclosure, there is provided a process for preparing a compound of formula (I), wherein R_{c} represents (i), or a pharmaceutically acceptable salt thereof, which comprises a deprotection reaction of a compound of formula (IV):

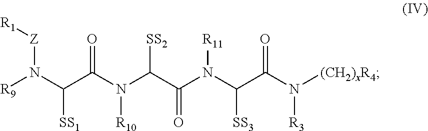

wherein Z, R₁, R₂, R₃, X, R₄, R₉, R₁₀, R₁₁, SS₁, SS₂ and SS₃ are as defined for the compound of formula (I);
wherein one or more of SS₁, SS₂ and SS₃ comprises a nucleophilic functional group and has a protecting group, such as StBu, on said functional group;
with a non-nucleophilic base, such as DIPEA and a reducing agent, such as DTT.

In an eleventh aspect of the disclosure, there is provided a process for preparing a compound of formula (IV) or a protected derivative thereof or salt thereof, which comprises reacting a compound of formula (V):

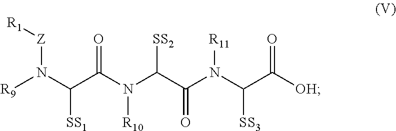

or a protected derivative thereof or a salt thereof;
with a compound of formula (VI):

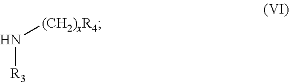

wherein Z, R₁, R₂, R₃, X, R₄, R₉, R₁₀, R₁₁, SS₁, SS₂ and SS₃ are as defined for the compound of formula (I).

### BRIEF DESCRIPTION OF THE FIGURE

FIG. 1 shows the fold-change in CAIX gene expression in cells treated with compounds no. 2, 13, 27, 28, 49 and 55 vs control (DMSO).

Detailed Description of Present Disclosure

### Definitions

As used herein, “alkyl” means a linear or branched alkane missing at least one hydrogen such that a bonding position is available, i.e. an alkyl group. Where a carbon chain length is not specified herein, “alkyl” means a C₁-C₁₀ alkyl group. In some embodiments, “alkyl” means a C₄-C₆ alkyl group. In other embodiments, “alkyl” means a C₁-C₃ alkyl group. Examples include methyl, ethyl, n-propyl and t-butyl. It may be monovalent, e.g. propyl, or divalent, e.g. propylene. A monovalent alkyl group may also be described by —CₙH₂ₙ₊₁ and a divalent alkyl group may also be described by —(CH₂)ₙ—, where n is independently selected from 1 to 10 for each substituent if not specified herein.

As used herein, “O-alkyl” means an alkyl group as defined above bonded to an oxygen atom, where said oxygen atom has a further available bonding position to form, for example, an ether via a C—O—C bond.

As used herein “halogen” or “halo” means an element from group 17 of the periodic table, preferably selected from fluorine, chlorine, bromine, and iodine, most preferably chlorine or fluorine, especially chlorine.

As used herein “haloalkyl” means an alkyl group as defined above, which may be substituted with up to 10 halogen atoms or more preferably up to 5 halogens. For example, they may be substituted by 1, 2, 3, 4 or 5 halogen atoms. Preferably, the halogen is fluorine. Preferably the haloalkyl is selected from —CF₃, —CHF₂, and —CH₂F, further preferably —CF₃.

As used herein “ring” means a monocyclic ring which can be aromatic or aliphatic (i.e. non-aromatic), a carbocycle or a heterocycle. “Aromatic” has its standard definition known in the art to be a conjugated system often made of alternating single and double bonds in a ring, for example a benzene or phenyl (Ph) ring. A “carbocycle” is a cyclic compound that has only carbon atoms forming the ring structure. “Heterocycle” or “Het” is a cyclic compound that has both carbon and non-carbon atoms forming the ring structure. Preferred non-carbon atoms (i.e. “heteroatoms”) are nitrogen, oxygen and sulphur. Preferably heterocycles contain one or two heteroatoms, preferably one. When there is more than one heteroatom in a heterocycle, the heteroatoms may be the same atom or different atoms.

Examples of suitable aliphatic carbocyclic rings typically contain 3 to 6 carbon ring members, such as cyclopropyl, cyclobutyl, cyclopentyl, cyclohexyl.

Examples of suitable aliphatic heterocyclic rings containing one or more heteroatoms selected from O, S and N include azetidine, pyrrolidine, piperidine, piperazine, morpholine, dioxane, tetrahydrofuran and thiomorpholine.

Examples of suitable aromatic heterocyclic rings containing one or more heteroatoms selected from O, S and N include furan, thiophene, pyrrole, imidazole, pyrazole, isoxazole, thiazole, isothiazole, pyridine, pyran, thiopyran, diazine, oxazine, thiazine, dioxine and dithiin.

Bicyclic rings are groups that feature two joined rings, they may be carbocyclic or heterocyclic, preferably heterocyclic, and aromatic or aliphatic, preferably aromatic. Preferably, the bicyclic rings are fused bicyclic compounds wherein the two rings share two adjacent atoms, i.e. the rings share one covalent bond. An example of a suitable aromatic bicyclic ring is benzothiophene.

Rings and bicyclic rings may optionally be substituted. Each of the rings and/or the bicyclic rings present on the compound may independently have zero, one, two or three substituents, preferably zero or one, more preferably one. A substituent may be on a carbon or a nitrogen atom.

Where an atom is identified herein, whether written or structurally indicated, said atom may be replaced by any known atomic isotopes of said atom, including stable and radioactive isotopes (i.e. variants of said atom differing in neutron number); for example, a deuterium atom may replace a hydrogen atom where a hydrogen atom is indicated. Synthetic methods for incorporating stable- and radio-isotopes are well-known in the art. Preferably, the atom is as identified herein.

As used herein, the above groups can be followed by the suffix -ene. This means that the group is divalent, i.e. a linker group. The linker (i.e. divalent) groups listed herein or in the claims are not ‘direction specific’. They can be reversed.

Compounds with which the disclosure is concerned, which may exist in one or more stereoisomeric form because of the presence of asymmetric atoms or rotational restrictions, can exist as a number of stereoisomers with R or S stereochemistry at each chiral centre or as atropisomers with R or S stereochemistry at each chiral axis. The disclosure includes all such enantiomers and diastereoisomers and mixtures thereof.

The skilled person will recognise that the compounds of the disclosure are short peptide chains consisting of three amino acids with modifications to the C and N caps on each end of the chain. Any references to the “compound(s)” of the disclosure are interchangeable with the “peptide(s)” of the disclosure or “tripeptide(s) of the disclosure”. It is well-known that amino acids have a side-chain attached to the central carbon atom. There are many known side-chains. The side-chains of the three amino acids in the peptides of the disclosure (represented by the symbol SS₁, SS₂ and SS₃) are variable. Structures and names of the side-chains that can be used in the present disclosure are shown in the table below:

| Name | Structure |
|---|---|
| leu | 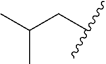 [SMILES: *CC(C)C] |
| h-leu | 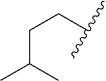 [SMILES: *CCC(C)C] |
| phe | 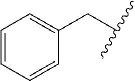 [SMILES: *Cc1ccccc1] |
| gly(Ph) | 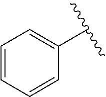 [SMILES: *c1ccccc1] |
| iso-leu | 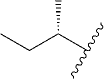 [SMILES: *[C@@H](C)CC] |
| allo-iso-leu | 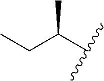 [SMILES: *[C@H](C)CC] |
| α-methyl- leu | 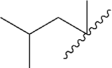 [SMILES: *CC(C)C] |
| nor-leu | 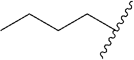 [SMILES: *CCCC] |
| cys | 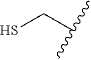 [SMILES: *CS] |
| h-cys | 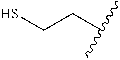 [SMILES: *CCS] |
| 2- pyridinethiol h-cys | 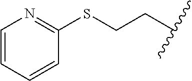 [SMILES: *CCSc1ccccn1] |
| methyl-cys | 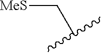 [SMILES: *CSC] |
| tyr(Ome) | 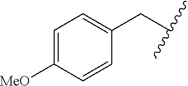 [SMILES: *Cc1ccc(OC)cc1] |
| h-phe | 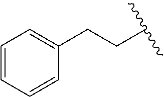 [SMILES: *CCc1ccccc1] |
| ala(1-naph) | 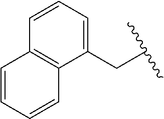 [SMILES: *Cc1cccc2ccccc12] |
| phe(RP) | 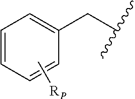 |
| | wherein the ring is |
| | substituted with one, |
| | two or three R_{P} groups; |
| | and |
| | R_{P} is independently I, |
| | Br, Cl, F, OH, Bz, NO₂, |
| | CN, or CF₃; |

Where a chemical structure is shown, the accuracy of the structure takes preference over the compound name.

### PREFERRED GROUPS OF THE DISCLOSURE

In a preferred embodiment, R₉, R₁₀, and/or R₁₁ are H, more preferably R₉, R₁₀, and R₁₁ are H.

Suitably one or more of R₉, R₁₀, or R₁₁ are methyl, preferably one of R₉, R₁₀, or R₁₁ is methyl. In one embodiment, R₉ is methyl and R₁₀ and R₁₁ are H. In an alternative embodiment, R₁₀ is methyl and R₉ and R₁₁ are H. In an alternative embodiment, Riu is methyl and R₉ and R₁₀ are H.

In a preferred embodiment Z represents SO₂. In an alternative embodiment Z represents CO. In an alternative embodiment Z represents H and there is no R₁ and R₉ is H, i.e. the NR₉ZR₁ group is simply an NH₂ group.

Suitably R₁ represents a straight-chain or branched C₁-C₈ alkyl, CH-(3-6 membered ring)₂, (CH₂)₁₋₃O(CH₂)₀₋₃CH₃, or (CH₂)₀₋₂R₂, preferably C₁-C₈ alkyl, CH-(3-6 membered ring)₂ or (CH₂)₀₋₂R₂, more preferably C₁-C₈ alkyl or (CH₂)₀₋₂R₂. Preferably, the C₁-C₈ alkyl is a C₁-C₆ alkyl, more preferably a C₁-C₄ alkyl. Preferably, the 3-6 membered ring is a phenyl ring. Preferably, the (CH₂)₁₋₃O(CH₂)₀₋₃CH₃ is (CH₂)₁O(CH₂)₁CH₃. Preferably, the (CH₂)₀₋₃R₂ is (CH₂)₀₋₂R₂.

Suitably R₁ represents a C₁ alkyl, a branched C₃ or C₄ alkyl, a straight-chain C₂ or C₃ alkyl, (CH₂)₁O(CH₂)₁CH₃, CHPh₂, (CH₂)₀R₂, (CH₂)₁R₂ or (CH₂)₂R₂.

Suitably R₂ represents NH(CH₂)₀₋₃CH₃; an 8-12 membered bicyclic ring optionally substituted by one or more of C₁-C₃ alkyl, HaI, CHHaI₂, CH₂HaI, and CHaI₃; or a 3-6 membered ring optionally substituted by one or more of C₁-C₃ alkyl, HaI, CHHaI₂, CH₂HaI, CHaI₃, CO(CH₂)₀₋₂CH₃, nitro group and (O)₀₋₁-3-6 membered ring optionally substituted by one or more of C₁-C₃ alkyl, HaI, CHHaI₂, CH₂HaI, and CHaI₃, preferably NH(CH₂)₀₋₁CH₃; an 8-10 membered bicyclic ring optionally substituted by one or more of C₁-C₃ alkyl and HaI; or a 3-6 membered ring optionally substituted by one or more of C₁-C₃ alkyl, HaI and (O)₀₋₁-3-6 membered ring optionally substituted by one or more of C₁-C₃ alkyl, HaI and CHaI₃.

Preferably, R₂ represents an 8-9 membered bicyclic ring, preferably a 9 membered bicyclic ring, optionally substituted by one or more of C₁-C₃ alkyl and HaI.

Alternatively, R₂ represents a 3-6 membered ring, preferably a 5 or 6 membered ring, more preferably an aromatic 5 or 6 membered ring. The 5 or 6 membered ring or the aromatic 5 or 6 membered ring may be optionally substituted by one or more of C₁-C₃ alkyl, HaI and (O)₀₋₁-3-6 membered ring optionally substituted by one or more of C₁-C₃ alkyl, HaI and CHaI₃; preferably HaI.

Preferably the (O)₀₋₁-3-6 membered ring is (O)₀₋₁-5-6 membered aromatic ring optionally substituted by one or more of C₁-C₃ alkyl, HaI and CHaI₃, preferably HaI.

Suitably R₂ represents NH(CH₂)₀CH₃, phenyl, furan, morpholine, cyclopropane, diphenyl ether, thiophene, 2-chlorothiophene, 1-methylimidazole, 2-bromothiophene, 2,3-dichlorothiophene, 2-chloro-3-nitrothiophene, 5-Chloro-3-methyl-1-benzothiophene, 5-(2-Thienyl)-1,2-oxazole or 1-methyl-5-thien-2-yl-3-(trifluoromethyl)-1H-pyrazole, preferably cyclopropane, 2-chlorothiophene, 1-methyl-5-thien-2-yl-3-(trifluoromethyl)-1H-pyrazole, 5-Chloro-3-methyl-1-benzothiophene, 2,3-dichlorothiophene, thiophene, diphenyl ether, phenyl, more preferably 2-chlorothiophene or 1-methyl-5-thien-2-yl-3-(trifluoromethyl)-1H-pyrazole.

In one preferred embodiment R_{c} is formula (i).

In a preferred embodiment R₃ represents H. In an alternative embodiment R₃ represents a straight-chain or branched C₁-C₈ alkyl, preferably a straight-chain or branched C₁-C₄ alkyl, more preferably a C₁-C₂ alkyl. Preferably the alkyls are straight-chain.

Suitably, X is an integer of from 0 to 8, preferably 0 to 6, more preferably 0 to 4, yet more preferably 0 to 3, especially 0 to 2, such as 0.

Suitably R₄ represents H; CHHaI₂; CH₂HaI; CHaI₃; HaI; CCH; NR₅R₆; CH(COOMe)(CH₂)₁₋₄(NH)C(NH₂)(NH); or 3-6 membered ring optionally substituted by one or more of C₁-C₃ alkyl, HaI, NR₇R₈ and COCH₃, preferably H; CHaI₃; HaI; CCH; NR₅R₆; CH(COOMe)(CH₂)₁₋₄(NH)C(NH₂)(NH); or 3-6 membered ring optionally substituted by one or more of C₁-C₃ alkyl, HaI, NR₇R₈ and COCH₃, more preferably H; HaI; or 3-6 membered ring optionally substituted by one or more of C₁-C₃ alkyl, HaI, NR₇R₈, and COCH₃, most preferably H or HaI.

Preferably R₄ represents the 3-6 membered ring, preferably a 5 or 6 membered ring, more preferably an aromatic 5 or 6 membered ring.

In one preferred embodiment R₅ represents H and R₆ represents a 3-6 membered ring, preferably a phenyl.

Suitably (CH₂)_{X}R₄ represents H, (CH₂)₁CH₃, (CH₂)₂CH₃, (CH₂)₁CF₃, (CH₂)₀-cyclopropane, (CH₂)₁CCH, (CH₂)₃Cl, (CH₂)₂NHPh, (CH₂)₅NH₂, (CH₂)₆NH₂, CH(COOMe)(CH₂)₃N(H)C(NH₂)(NH), (CH₂)₀-tetrahydropyran-4-yl, (CH₂)₁Ph, (CH₂)₁-furan-2-yl, (CH₂)₁-thiophen-2-yl or (CH₂)₁-pyridin-2-yl, preferably H, (CH₂)₃Cl, (CH₂)₂CH₃, (CH₂)₁Ph or (CH₂)₁-thiophen-2-yl, more preferably H or (CH₂)₃Cl.

In one particular embodiment, R₃ represents H, X represents 0 and R₄ represents H.

In one embodiment R_{c} is formula (ii). In another embodiment R_{c} is formula (iii).

Preferably each RP independently represents I, Br, Cl, F, OH, Bz, NO₂, CN, or CF₃, more preferably I, Br, Cl, F, OH, and even more preferably I or OH, especially I.

In one aspect it may be that SS₁, SS₂ and SS₃ are independently selected from leu, iso-leu, allo-iso-leu, h-leu, nor-leu, phe, phe(R_{P}), h-phe, gly(Ph), tyr(OMe), cys, h-cys or methyl-cys, more preferably h-leu, nor-leu, phe, phe(R_{P}), h-phe, cys or h-cys, and more preferably h-leu, phe, phe(Rp) or cys.

SS₁, SS₂ and SS₃ are independently selected from one of the side-chains in list (a), list (b) or list (c):

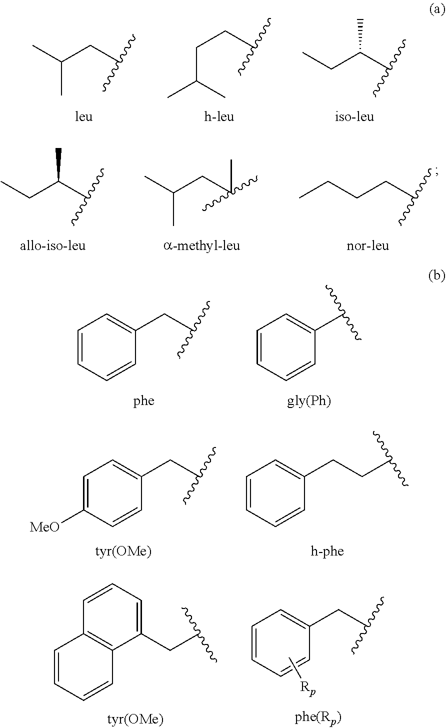

wherein the ring of phe(Rp) is substituted with one, two, or three R_{P} groups, and each R_{P} is independently I, Br, CI, F, OH, Bz, NO₂, CN, or CF₃;

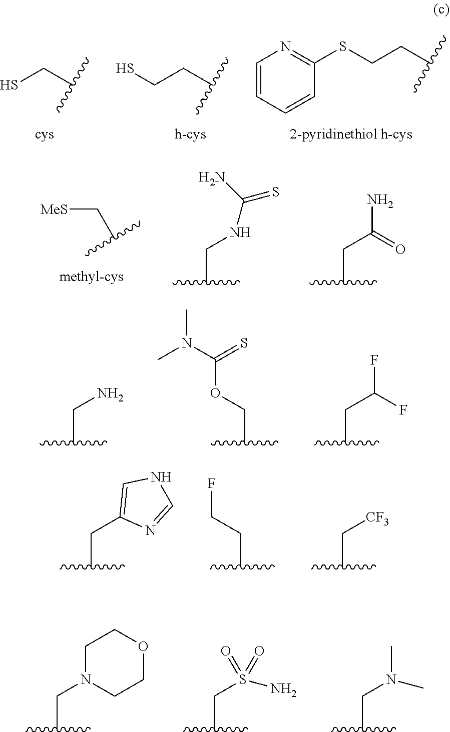

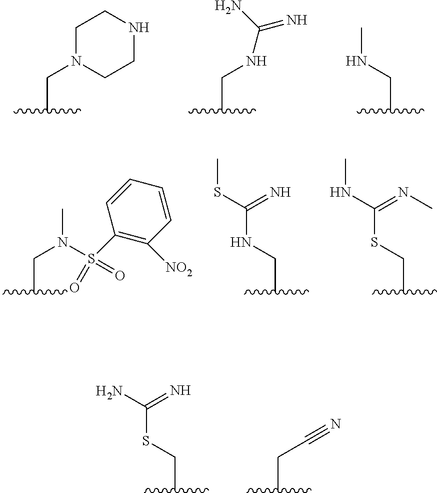

wherein SS₁, SS₂ and SS₃ are side-chains each selected from different lists. Preferably, SS₁ is selected from (a), SS₂ is selected from (b) and SS₃ is selected from (c).

Preferably, (a) represents leu, iso-leu, allo-iso-leu, h-leu or nor-leu, more preferably h-leu or nor-leu, and more preferably h-leu.

Preferably, (b) represents phe, phe(R_{P}), h-phe, gly(Ph) or tyr(OMe), more preferably phe, phe(R_{P}) or h-phe, even more preferably phe or phe(R_{P}).

Preferably, (c) represents cys, h-cys, 2-pyridinethiol h-cys or methyl-cys; more preferably cys, h-cys or methyl-cys; more preferably cys or h-cys, most preferably cys.

In an embodiment, the ring of phe(R_{P}), is substituted with one R_{P} group. The one R_{P} group may be at the para-position; i.e. phe(4-R_{P}), which is one of the following:

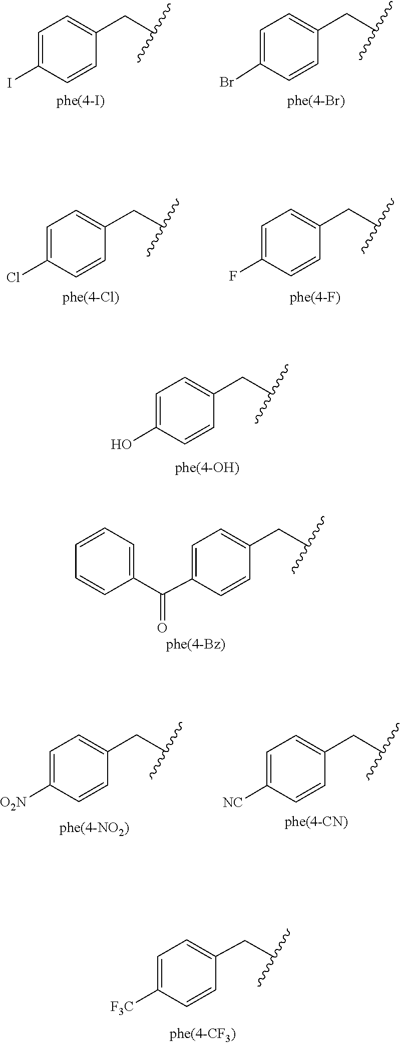

In an alternative embodiment, the one R_{P} group is at the meta-position; i.e. phe(3-R_{P}), which is one of the following:

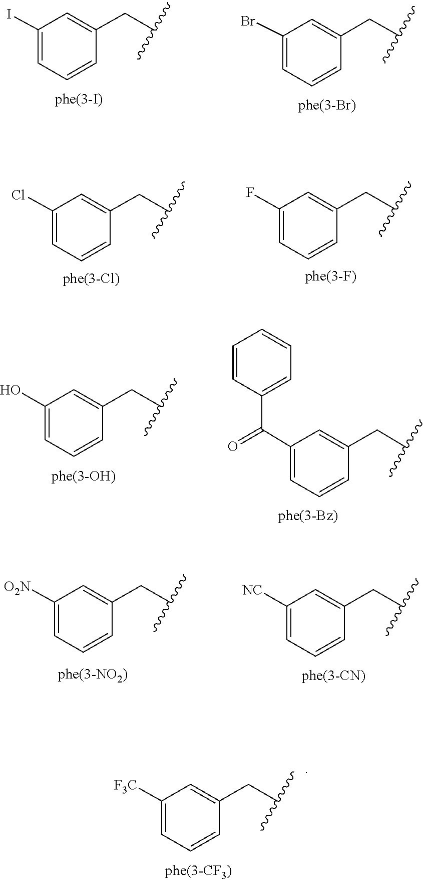

In an alternative embodiment, the one R_{P} group is at the ortho-position; i.e. phe(2-R_{P}), which is one of the following:

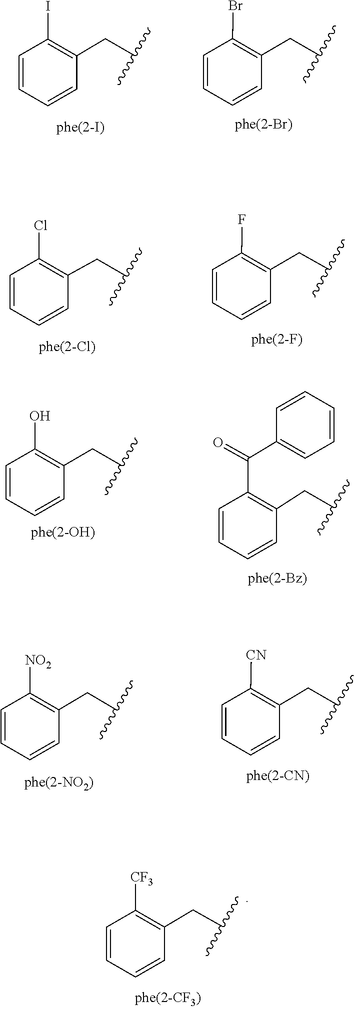

In an alternative embodiment, the ring of phe(R_{P}) is substituted with two R_{P} groups; i.e. phe(R_{P}) is one of the following:

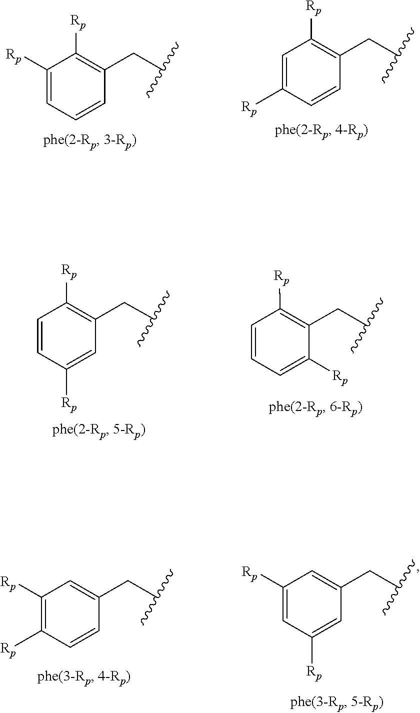

wherein R_{P} is independently: I, Br, Cl, F, OH, Bz, NO₂, CN, or CF₃;
preferably at least one R_{P} is I, Br, Cl, F or OH; more preferably, at least one R_{P} is I.

In an alternative embodiment, the ring of phe(R_{P}) is substituted with three R_{P} groups; i.e phe(R_{P}) is one of the following:

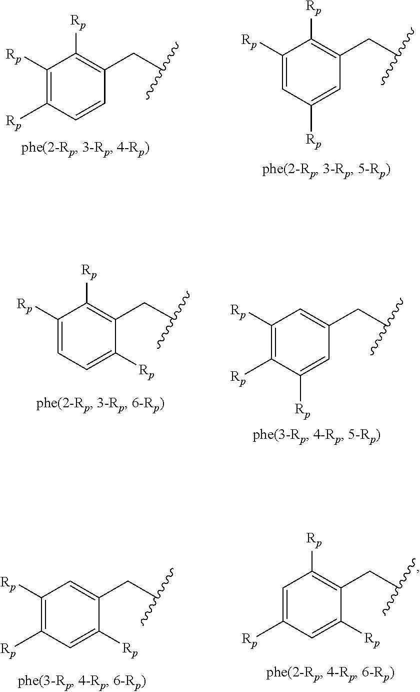

wherein R_{P} is independently: I, Br, Cl, F, OH, Bz, NO₂, CN, or CF₃;
preferably at least one R_{P} is I, Br, Cl, F or OH; more preferably, at least one R_{P} is I.

Where appropriate, the definitions and preferences described above are applicable to the following specific embodiments. In one specific embodiment the disclosure provides the compound of formula (I),
wherein Z═CO or SO₂ or H, wherein if Z is H there is no R₁;
R₉, R₁₀ and R₁₁ are independently H or methyl;
R₁=straight-chain or branched C₁-C₈alkyl; CH-(3-6 membered ring)₂; (CH₂)₁₋₃O(CH₂)₀₋₃CH₃; or (CH₂)₀₋₃R₂;
R₂═NH(CH₂)₀₋₃CH₃; a 8-12 membered bicyclic ring optionally substituted by one or more of C₁-C₃ alkyl, HaI, CHHaI₂, CH₂HaI, and CHaI₃; or a 3-6 membered ring optionally substituted by one or more of C₁-C₃ alkyl, HaI, and (O)₀₋₁-3-6 membered ring optionally substituted by one or more of C₁-C₃ alkyl, HaI, CHHaI₂, CH₂HaI, and CHaI₃;
R_{c} is (i), or (iii):
wherein (i) is

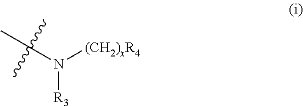

wherein R₃═H; or straight-chain or branched C₁-C₄ alkyl;
X=an integer of from 0 to 8;
R₄═H; CH₃; CHHaI₂; CH₂HaI; CHaI₃; HaI; CCH; NR₅R₆; CH(COOMe)(CH₂)₁₋₄NHC(NH₂)(NH); or 3-6 membered ring optionally substituted by one or more of C₁-C₃ alkyl, HaI, NR₇R₈, and COCH₃;
R₅═H; or straight-chain or branched C₁-C₃ alkyl;
R₆═H; or straight-chain or branched C₁-C₃ alkyl; or 3-6 membered ring;
R₇═H; or straight-chain or branched C₁-C₃ alkyl;
R₈═H; or straight-chain or branched C₁-C₃ alkyl;
wherein (iii) is OH;
wherein HaI is a halogen;
wherein SS₁, SS₂ and SS₃ are independently selected from one of the side-chains in list (a), list (b), or list (c):
(a) h-leu, leu, iso-leu, allo-iso-leu, α-methyl-leu, nor-leu;
(b) phe, h-phe, ala(1-naph), gly(Ph), tyr(OMe), phe(R_{P});
(c) cys, h-cys, 2-pyridinethiol h-cys or methyl-cys or one of the side-chains pictured below:

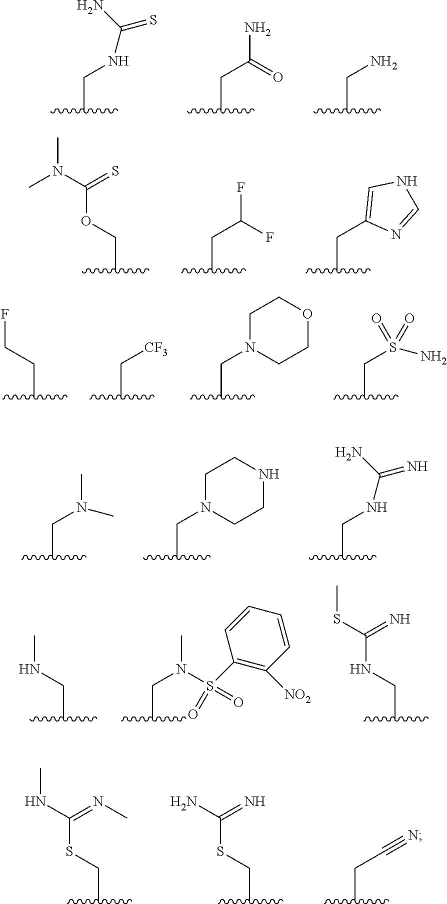

wherein SS₁, SS₂ and SS₃ are each selected from different lists;
or a pharmaceutically acceptable salt thereof. Preferably list (c) is cys, h-cys, 2-pyridinethiol h-cys or methyl-cys.

In one specific embodiment the disclosure provides the compound of formula (I),
wherein Z═CO or SO₂ or H, wherein if Z is H there is no R₁;
R₉, R₁₀ and R₁₁ are independently H or methyl;
R₁=straight-chain or branched C₁-C₈ alkyl; CH-(3-6 membered ring)₂; or (CH₂)₀₋₃R₂;
R₂=a 8-12 membered bicyclic ring optionally substituted by one or more of C₁-C₃ alkyl, HaI; or a 3-6 membered ring optionally substituted by one or more of C₁-C₃ alkyl, HaI, and (O)₀₋₁-3-6 membered ring optionally substituted by one or more of C₁-C₃ alkyl, and CHaI₃;
R_{C} is (i), or (iii):
wherein (i) is

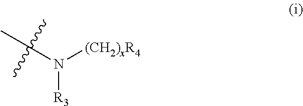

wherein R₃═H; or straight-chain or branched C₁-C₄ alkyl;
X=an integer of from 0 to 4;
R₄═H; CH₃; HaI; or 3-6 membered ring optionally substituted by one or more of C₁-C₃ alkyl, HaI, NR₇R₈, and COCH₃;
R₇═H; or straight-chain or branched C₁-C₃ alkyl;
R₈═H; or straight-chain or branched C₁-C₃ alkyl;
wherein (iii) is OH;
wherein HaI is a halogen;
wherein SS₁, SS₂ and SS₃ are independently selected from one of the side-chains in list (a), list (b), or list (c):
(a) h-leu, leu, iso-leu, allo-iso-leu, α-methyl-leu, nor-leu;
(b) phe, h-phe, ala(1-naph), gly(Ph), tyr(OMe), phe(R_{P});
(c) cys, h-cys, 2-pyridinethiol h-cys or methyl-cys or one of the side-chains pictured below:

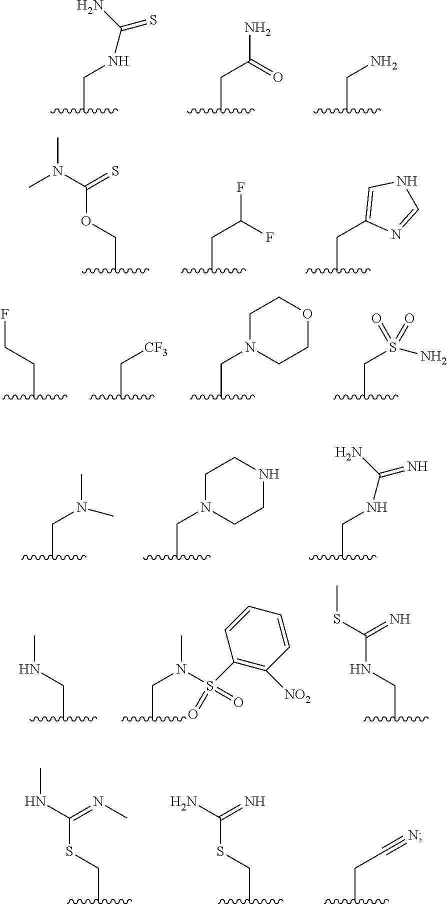

wherein SS₁, SS₂ and SS₃ are each selected from different lists;
or a pharmaceutically acceptable salt thereof. Preferably list (c) is cys, h-cys, 2-pyridinethiol h-cys or methyl-cys.

In one specific embodiment the disclosure provides the compound of formula (I),
wherein Z═CO or SO₂ or H, wherein if Z is H there is no R₁;
R₉, R₁₀ and R₁₁ are H;
R₁=straight-chain or branched C₁-C₈ alkyl; CH-(3-6 membered ring)₂; (CH₂)₁₋₃O (CH₂)₀₋₃CH₃; or (CH₂)₀₋₃R₂;
R₂═NH(CH₂)₀₋₃CH₃; a 8-12 membered bicyclic ring optionally substituted by one or more of C₁-C₃ alkyl, HaI, CHHaI₂, CH₂HaI, and CHaI₃; or a 3-6 membered ring optionally substituted by one or more of C₁-C₃ alkyl, HaI, CHHaI₂, CH₂HaI, CHaI₃, CO(CH₂)₀₋₂CH₃, nitro group and (O)₀₋₁-3-6 membered ring optionally substituted by one or more of C₁-C₃ alkyl, HaI, CHHaI₂, CH₂HaI, and CHaI₃;
R_{C} is (i), (ii) or (iii):
wherein (i) is:

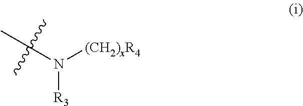

wherein R₃═H; or straight-chain or branched C₁-C₈ alkyl;
X=an integer of from 0 to 8;
R₄═H; CHHaI₂; CH₂HaI; CHaI₃; HaI; CCH; NR₅R₆; CH(COOMe)(CH₂)₁₋₄NHC(NH₂)(NH);
or 3-6 membered ring optionally substituted by one or more of C₁-C₃ alkyl, HaI, NR₇R₈, and COCH₃;
R₅═H; or straight-chain or branched C₁-C₃ alkyl;
R₆═H; or straight-chain or branched C₁-C₃ alkyl; or 3-6 membered ring;
R₇═H; or straight-chain or branched C₁-C₃ alkyl;
R₈═H; or straight-chain or branched C₁-C₃ alkyl;
wherein (ii) is a 5-6 membered heterocycle comprising at least one nitrogen atom and optionally substituted by one or more of C₁-C₃ alkyl, HaI, CHHaI₂, CH₂HaI, CHaI₃, NR₇R₈, and COCH₃, wherein R_{c} is bonded via the at least one nitrogen atom;
wherein (iii) is OH;
wherein HaI is a halogen;
wherein SS₁, SS₂ and SS₃ are independently selected from one of the side-chains in list (a), list (b), or list (c):
(a) h-leu, leu, iso-leu, allo-iso-leu, α-methyl-leu, nor-leu;
(b) phe, h-phe, ala(1-naph), gly(Ph), tyr(OMe), phe(R_{P});
(c) cys, h-cys, 2-pyridinethiol h-cys or methyl-cys;
wherein SS₁, SS₂ and SS₃ are each selected from different lists; or a pharmaceutically acceptable salt thereof.

The compounds of formula (I) wherein R_{c} represents (i), R₃ represents H, X represents 0 and R₄ represents H, may conveniently be prepared by a process comprising removing the compound of formula (II) from a rink-amide resin and removing any protecting groups, wherein formula (II) is:

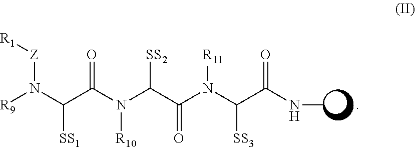

Suitably the removal and deprotection occurs in the presence of a strong acid such as TFA and a scavenger, such as TIS, in water at room temperature. The skilled person will be aware of functional groups that need protecting in order for the desired reaction described above to occur. Such functional groups may be protected by well-known protecting groups by well-known methods.

The compounds of formula (II) may conveniently be prepared by a process comprising reacting a compound of formula (III):

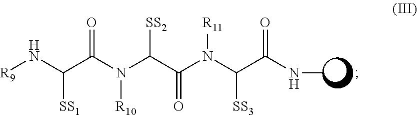

with a compound of R₁ZCl. Suitably the reaction occurs in the presence of a non-nucleophilic base, such as DIPEA, and a solvent, such as DMF, at room temperature for approximately 30 minutes. The skilled person will recognise when a protecting group is required on any of SS₁, SS₂ and SS₃, for example, if cys is any of SS₁, SS₂ or SS₃ i.e. there is a sulphide present, then a protecting group, such as StBu, will be required.

The compounds of formula (III) may conveniently be prepared by the well-known standard process in the field; SPPS.

The compounds of formula (I) wherein R_{c} represents (i) may conveniently be prepared by a deprotection reaction comprising reacting a compound of formula (IV):

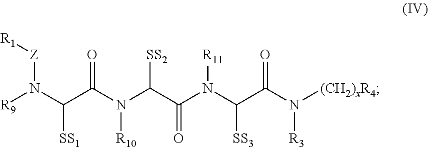

wherein one or more of SS₁, SS₂ and SS₃ comprises a nucleophilic functional group and has a protecting group, such as StBu, on said functional group;
with a non-nucleophilic base, such as DIPEA and a reducing agent, such as DTT, in a solvent, such as DMF, at room temperature for approximately an hour.

Suitably the nucleophilic functional group is a sulphide.

The compounds of formula (IV) may conveniently be prepared by a process comprising reacting a compound of formula (V):

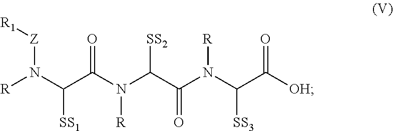

with a compound of formula (VI):

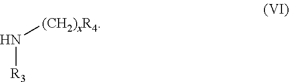

Suitably the reaction between compounds of formulas (V) and (VI) occurs in the presence of a non-nucleophilic base, such as DIPEA, and a solvent, such as DMF, at room temperature for approximately an hour. The skilled person will recognise when a protecting group is required on any of SS₁, SS₂ and SS₃, for example, if cys is any of SS₁, SS₂ or SS₃ i.e. there is a sulphide present, then a protecting group, such as StBu, will be required.

The compounds of formula (V) may conveniently be prepared by a process comprising removing the compound of formula (VII) from a 2-chlorotrityl resin, wherein the compound of formula (VII) is:

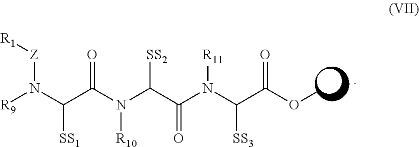

Suitably the removal occurs in a solvent mixture, such as a polar solvent like DCM, and a hard Lewis acid, such as HFIP, suitably at a 4:1 ratio and at room temperature for approximately 2.5 hours.

The compounds of formula (VII) may conveniently be prepared by a process comprising reacting a compound of formula (VIII):

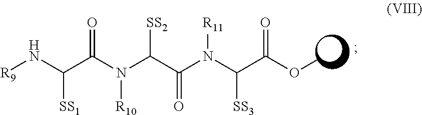

with a compound of R₁ZCl. Suitably the reaction occurs in the presence of a non-nucleophilic base, such as DIPEA, and a solvent, such as DMF, at room temperature for approximately 30 minutes. The skilled person will recognise when a protecting group is required on any of SS₁, SS₂ and SS₃, for example, if cys is any of SS₁, SS₂ or SS₃ i.e. there is a sulphide present, then a protecting group, such as StBu, will be required.

The compounds of formula (VIII) may conveniently be prepared by the well-known standard process in the field; SPPS. Compounds of formula (VI) may be prepared by known methods.

Novel intermediates including compounds of formula (II), (IV), (V) and (VII) and salts thereof are claimed as an aspect of the disclosure.

Compounds of formula (I) may be prepared or employed in the form of a pharmaceutically acceptable salt, including the therapeutically active non-toxic acid addition salts that the compounds of formula (I) are able to form. These pharmaceutically acceptable acid addition salts can conveniently be obtained by treating the free base form with such appropriate acids in a suitable solvent or mixture of solvents. Appropriate acids comprise, for example, ethanesulfonic, maleic, malonic, L-tartaric, fumaric, citric, succinic, acetic, triphenyl acetic, hydrochloric, sulfuric, phosphoric, 1-hydroxy-2-naphthoic, hydrobromic, methanesulfonic, tartaric, palmitic, isethionic, pamoic, formic, cinnamic benzoic, ascorbic, galactaric, lactic, malic, oxalic, para-toluenesulfonic, benzenesulphonic, propionic, furoic, phosphonic and glutaric. Conversely said salt forms can be converted by treatment with an appropriate base into the free base form.

As described above, the peptides of the present disclosure are considered to be useful in inhibiting the interaction between HIF-α and HIf-1 β.

Accordingly, in one embodiment the peptide is an inhibitor of the interaction between HIF-2α and HIF-1β. In one embodiment the peptide is capable of binding to HIF-2α, for example is capable of binding to a recombinantly expressed PAS-B domain of HIF-2α.

In another or the same embodiment, the peptide is an inhibitor of the interaction between HIF-1α and HIF-1β. In one embodiment the peptide is capable of binding to HIF-1α, for example is capable of binding to recombinantly expressed PAS-B domain of HIF-1α.

It is preferred if the peptide is both an inhibitor of the interaction between HIF-2α and HIF-1β and of the interaction between HIF-1α and HIF-1β. Accordingly, in some embodiments the peptide is capable of binding to HIF-1α and HIF-2α, for example is capable of binding to recombinantly expressed PAS-B domain of HIF-2α and HIF-1α.

The peptide may bind to any region of HIF-1α and/or HIF-2α. As described in the examples, in some embodiments the peptide binds to recombinantly expressed PAS-B domain of HIF-2α and or recombinantly expressed PAS-B domain of HIF-1α.

It is even more preferred if the peptide binds to HIF-1α and HIF-2α with no or little bias, i.e. binds to HIF-1α and HIF-2α with the same or similar affinity.

The skilled person will recognise that there are multiple means to determine the binding affinity of the peptide to HIF-1α and HIF-2α or to recombinantly expressed PAS-B domain of both HIF-1α and 2α. For example, in one embodiment the affinity of determined using microscale thermophoresis, for example is determining against the recombinantly expressed PAS-B domain of both HIF-1α and 2α using microscale thermophoresis.

The skilled person will understand when the affinity to which the peptide binds to HIF-1α and the affinity with which the peptide binds to HIF-2α is sufficiently similar to render the peptide particular useful. In one embodiment the peptide binds to HIF-1α and HIF-2α with a similar affinity when the difference in affinity of binding to HIF-1 α and HIF-2α is:
less than 60 μM, 55 μM, 50 μM, 45 μM, 40 μM, 35 μM, 30 μM, 25 μM, 20 μM, 18 μM, 16 μM, 15 μM, 14 μM, 13 μM, 12 μM, 11 μM, 10 μM, 9 μM, 8 μM, 7 μM, 6 μM, 5 μM, 4 μM, 3 μM, 2 μM, 1 μM, 0.8 μM, 0.6 μM, 0.5 μM, 0.4 μM, 0.3 μM, 0.2 μM, 0.1 μM; and/or
between 0.1 μM and 10 μM, 0.2 μM and 9 μM, 0.3 μM and 8 μM, 0.4 μM and 7 μM, 0.5 μM and 6 μM, 0.6 and 5.5 μM, 0.7 and 5 μM, 0.8 and 4.5 μM, 0.9 μM and 4 μM, 1 and 3.5 μM, 1.25 μM and 3 μM, 1.5 μM and 2.75 μM, 1.75 μM and 2.5 μM, 2 μM and 2.25 μM; and/or
between 0.1 μM and 60 μM, 0.2 μM and 55 μM, 0.3 μM and 50 μM, 0.4 μM and 45 μM, 0.5 μM and 40 μM, 0.6 μM and 35 μM, 0.7 μM and 30 μM, 0.8 μM and 25 μM, 0.9 μM and 20 μM, 1 μM and 18 μM, 2 μM and 16 μM, 3 μM and 15 μM, 4 μM and 14 μM, 5 μM and 13 μM, 6 μM and 12 μM, 7 μM and 11 μM, 8 μM and 10 μM.

As described above, the ability of a peptide to bind to HIF-1α and HIF-2α can be determined by determining the ability of the peptide to bind to recombinantly expressed PAS-B domain of HIF-1α and HIF-2α. Accordingly, in one embodiment the peptide binds to recombinantly expressed PAS-B domain of HIF-2α and HIF-1α with a similar affinity when the difference in affinity of binding to recombinantly expressed PAS-B domain of HIF-2α and HIF-1α is:
less than 60 μM, 55 μM, 50 μM, 45 μM, 40 μM, 35 μM, 30 μM, 25 μM, 20 μM, 18 μM, 16 μM, 15 μM, 14 μM, 13 μM, 12 μM, 11 μM, 10 μM, 9 μM, 8 μM, 7 μM, 6 μM, 5 μM, 4 μM, 3 μM, 2 μM, 1 μM, 0.8 μM, 0.6 μM, 0.5 μM, 0.4 μM, 0.3 μM, 0.2 μM, 0.1 μM; and/or between 0.1 μM and 10 μM, 0.2 μM and 9 μM, 0.3 μM and 8 μM, 0.4 μM and 7 μM, 0.5 μM and 6 μM, 0.6 and 5.5 μM, 0.7 and 5 μM, 0.8 and 4.5 μM, 0.9 μM and 4 μM, 1 and 3.5 μM, 1.25 μM and 3 μM, 1.5 μM and 2.75 μM, 1.75 μM and 2.5 μM, 2 μM and 2.25 μM; and/or
between 0.1 μM and 60 μM, 0.2 μM and 55 μM, 0.3 μM and 50 μM, 0.4 μM and 45 μM, 0.5 μM and 40 μM, 0.6 μM and 35 μM, 0.7 μM and 30 μM, 0.8 μM and 25 μM, 0.9 μM and 20 μM, 1 μM and 18 μM, 2 μM and 16 μM, 3 μM and 15 μM, 4 μM and 14 μM, 5 μM and 13 μM, 6 μM and 12 μM, 7 μM and 11 μM, 8 μM and 10 μM,
for example, wherein the affinity is determined against the recombinantly expressed PAS-B domain of both HIF-1α and 2a using microscale thermophoresis.

In some embodiments, the peptide binds to recombinantly expressed PAS-B domain of HIF-1α with an affinity of:
less than 10 μM, 9.5 μM, 9 μM, 8.5 μM, 8 μM, 7.5 μM, 7 μM, 6.5 μM, 6 μM, 5.5 μM, 5 μM, 4.5 μM, 4 μM, 3.5 μM, 3 μM, 2.5 μM, 2 μM, 1.5 μM, 1 μM, 0.9 μM, 0.8 μM, 0.7 μM, 0.6 μM, 0.5 μM, 0.4 μM, 0.3 μM, 0.2 μM, 0.1 μM; and/or
between 0.1 μM and 10 μM, 0.2 μM, and 9.5 μM, 0.3 μM and 9 μM, 0.4 μM and 8.5 μM, 0.5 μM and 8 μM, 0.6 μM and 7.5 μM, 0.7 μM and 7 μM, 0.8 μM and 6.5 μM, 0.9 μM and 6 μM, 1 μM and 5.5 μM, 1.5 μM and 5 μM, 2 μM and 5 μM, 2.5 μM and 4.5 μM, 3 μM and 4 μM; and/or
between 0.5 μM and 5 μM, 0.6 μM and 4.5 μM, 0.7 μM and 4 μM, 0.8 μM and 3.5 μM, 0.9 μM and 3 μM, 1 μM and 2.5 μM, 1.25 μM and 2.25 μM, 1.5 μM and 2 μM,
for example, wherein the affinity is determined against the recombinantly expressed PAS-B domain of HIF-1α using microscale thermophoresis.

In some embodiments, the peptide binds to recombinantly expressed PAS-B domain of HIF-2α with an affinity of:
less than 10 μM, 9.5 μM, 9 μM, 8.5 μM, 8 μM, 7.5 μM, 7 μM, 6.5 μM, 6 μM, 5.5 μM, 5 μM, 4.5 μM, 4 μM, 3.5 μM, 3 μM, 2.5 μM, 2 μM, 1.5 μM, 1 μM, 0.9 μM, 0.8 μM, 0.7 μM, 0.6 μM, 0.5 μM, 0.4 μM, 0.3 μM, 0.2 μM, 0.1 μM; and/or
between 0.1 μM and 10 μM, 0.2 μM, and 9.5 μM, 0.3 μM and 9 μM, 0.4 μM and 8.5 μM, 0.5 μM and 8 μM, 0.6 μM and 7.5 μM, 0.7 μM and 7 μM, 0.8 μM and 6.5 μM, 0.9 μM and 6 μM, 1 μM and 5.5 μM, 1.5 μM and 5 μM, 2 μM and 5 μM, 2.5 μM and 4.5 μM, 3 μM and 4 μM; and/or
between 0.5 μM and 5 μM, 0.6 μM and 4.5 μM, 0.7 μM and 4 μM, 0.8 μM and 3.5 μM, 0.9 μM and 3 μM, 1 μM and 2.5 μM, 1.25 μM and 2.25 μM, 1.5 μM and 2 μM,
for example, wherein the affinity is determined against the recombinantly expressed PAS-B domain of HIF-2a using microscale thermophoresis.

In some embodiments, the peptide binds to recombinantly expressed PAS-B domain of both HIF-1α and HIF-2α with an affinity of:
less than 10 μM, 9.5 μM, 9 μM, 8.5 μM, 8 μM, 7.5 μM, 7 μM, 6.5 μM, 6 μM, 5.5 μM, 5 μM, 4.5 μM, 4 μM, 3.5 μM, 3 μM, 2.5 μM, 2 μM, 1.5 μM, 1 μM, 0.9 μM, 0.8 μM, 0.7 μM, 0.6 μM, 0.5 μM, 0.4 μM, 0.3 μM, 0.2 μM, 0.1 μM; and/or
between 0.1 μM and 10 μM, 0.2 μM, and 9.5 μM, 0.3 μM and 9 μM, 0.4 μM and 8.5 μM, 0.5 μM and 8 μM, 0.6 μM and 7.5 μM, 0.7 μM and 7 μM, 0.8 μM and 6.5 μM, 0.9 μM and 6 μM, 1 μM and 5.5 μM, 1.5 μM and 5 μM, 2 μM and 5 μM, 2.5 μM and 4.5 μM, 3 μM and 4 μM; and/or
between 0.5 μM and 5 μM, 0.6 μM and 4.5 μM, 0.7 μM and 4 μM, 0.8 μM and 3.5 μM, 0.9 μM and 3 μM, 1 μM and 2.5 μM, 1.25 μM and 2.25 μM, 1.5 μM and 2 μM,
for example, wherein the affinity is determined against the recombinantly expressed PAS-B domain of both HIF-1α and 2a using microscale thermophoresis.

The peptides of the disclosure are useful in preventing or reducing the response to hypoxia. Accordingly, in one embodiment the peptide of the disclosure prevents or reduces the hypoxia induced expression from a promoter that comprises one or more hypoxia-responsive elements under hypoxic conditions.

Hypoxia is a state of reduced oxygen concentration that can arise under normal conditions such as embryonic development, and in, for example, the tumour microenvironment.

In one embodiment, the peptide reduces the hypoxia induced expression from a promoter that comprises one or more hypoxia-responsive elements under hypoxic conditions to less than 90%, 80%, 70%, 60%, 50%, 40%, 30%, 20%, 10%, 5%, 4%, 3%, 2%, 1% of the expression obtained in the absence of the peptide under hypoxic conditions. For example in one embodiment, the peptide reduces the hypoxia induced expression of a reporter protein, for example YFP, from a promoter that comprises one or more hypoxia-responsive elements, for example from a promoter that comprises three HRE sequences, under hypoxic conditions.

In one embodiment the peptides of the disclosure can disrupt the interaction between HIF-1α and HIF-1β, i.e. prevent association or binding of the α and β subunits of the HIF heterodimeric protein. In the same or different embodiment, the peptides of the disclosure can disrupt the interaction between HIF-2α and HIF-1β. In a preferred embodiment, the peptides are able to disrupt the interaction between HIF-1α and HIF-1β, and between HIF-2α and HIF-1β.

Various methods are used to determine protein-protein interactions, including yeast two hybrid assays and protein cross linking methods. The proximity ligation assay may also be used, whereby the interacting partner domains are targeted by separate primary and secondary antibodies. The secondary antibodies comprise PLA probes that contain short DNA strands. When in close proximity, i.e. where the interacting partner domains are contacting one another, the DNA strands can be amplified via rolling circle DNA synthesis. Identification of an amplification product indicates an interaction between the two partner domains. The proximity ligation assay can be performed in situ, for example in cells, for example in MCF-7 cells.

Accordingly, in one embodiment the peptide disrupts the interaction between HIF-1α and HIF-1β; between HIF-2α and HIF-1β; or between HIF-1α and HIF-1β, and between HIF-2α and HIF-1β, wherein the interaction is assessed by proximity ligation assay, optionally in MCF-7 cells.

Since the peptides of the disclosure are able to disrupt the typical response to hypoxia, it will be apparent to the skilled person that the peptides of the disclosure have use in the treatment and/or prevention of diseases, disorders or conditions. For example, in one embodiment the peptides of the disclosure are useful in the treatment or prevention of a disease, disorder or condition that experiences a hypoxic environment and requires the typical hypoxia response for maintenance. The peptides of the disclosure are also suitable for treatment or prevention of any other disease treatable or preventable by inhibition of dimerization of HIF-1a with HIF1-b and HIF2a with HIF1b and/or inhibits the activity of HIF-1 and HIF-2 and/or HIF-1 or HIF-2 signalling. The peptides of the disclosure are also suitable for use in the treatment or prevention of a disease, disorder or condition in which it is desirable to repress hypoxia induced gene expression.

Such diseases, disorders and conditions include Von Hippel-Lindau disease, tumours and cancer.

A tumour is not necessarily the same as cancer. By tumour we include the meaning of any kind of aberrant growth, whether it is benign or malignant. By cancer we include solid cancers and blood cancers. Solid cancers typically refer to an aberrant growth that is or has the potential to be malignant. Blood cancers are not solid cancers and include, for example, lymphomas.

Solid tumours and solid cancers in particular are known to experience a hypoxic tumour microenvironment, and it is known that a hypoxic tumour microenvironment correlates with poor prognosis. Blocking the response to hypoxia using the peptides of the disclosure is considered to be useful in the treatment and/or prevention of these diseases, disorders and conditions.

Accordingly, in one embodiment the cell is a human cell. In yet another embodiment the cell is a diseased cell, for example is a cancer cell. In one embodiment the cell is an in vitro cell, such as an in vitro mammalian cell or in vitro human cell. For example, in vitro human cells comprising the peptide of the disclosure may be used as part of a screening procedure to determine appropriate treatment strategies. In some embodiments the cell is not an in vivo human cell. In some embodiment the cell is not an in vivo human or animal cell.

Accordingly, the disclosure provides a pharmaceutical composition comprising one or more of the peptides of the disclosure of the disclosure.

As used herein, “pharmaceutical composition” means a therapeutically effective formulation for use in the treatment or prevention of the diseases, disorders and/or conditions described herein.

Examples of such diseases, disorders and conditions includes cancer, such as solid cancer, or Von Hippel-Lindau disease.

Additional compounds may also be included in the pharmaceutical compositions, such as other peptides, low molecular weight immunomodulating agents, receptor agonists and antagonists, and antimicrobial agents. Other examples include chelating agents such as EDTA, citrate, EGTA or glutathione.

The pharmaceutical compositions may be prepared in a manner known in the art that is sufficiently storage stable and suitable for administration to humans and animals. The pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the biological activity of the active ingredients, i.e. the peptides of the disclosure. Such pharmaceutically acceptable buffers, carriers, diluents or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), which are incorporated herein by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the peptide in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the peptide of the composition. The adjuvant may be one or more of colloidal silver, or zinc, copper or silver salts with different anions, for example, but not limited to fluoride, chloride, bromide, iodide, tiocyanate, sulfite, hydroxide, phosphate, carbonate, lactate, glycolate, citrate, borate, tartrate, and acetates of different acyl composition. The adjuvant may also be cationic polymers such as PHMB, cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, sucrose, mannitol, and cyclodextrines, which are added to the composition, e.g., for facilitating lyophilisation. Examples of polymers are starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, carageenans, hyaluronic acid and derivatives thereof, polyacrylic acid, polysulphonate, polyethylenglycol/polyethylene oxide, polyethyleneoxide/polypropylene oxide copolymers, polyvinylalcohol/polyvinylacetate of different degree of hydrolysis, poly(lactic acid), poly(glycholic acid) or copolymers thereof with various composition, and polyvinylpyrrolidone, all of different molecular weight, which are added to the composition, e.g. for viscosity control, for achieving bioadhesion, or for protecting the active ingredient (applies to A-C as well) from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The pharmaceutical composition may also contain one or more mono- or di-saccharides such as xylitol, sorbitol, mannitol, lactitiol, isomalt, maltitol or xylosides, and/or monoacylglycerols, such as monolaurin. The characteristics of the carrier are dependent on the route of administration. One route of administration is topical administration. For example, for topical administrations, a preferred carrier is an emulsified cream comprising the active peptide, but other common carriers such as certain petrolatum/mineral-based and vegetable-based ointments can be used, as well as polymer gels, liquid crystalline phases and microemulsions.

It will be appreciated that the pharmaceutical compositions may comprise one or more of the peptides of the disclosure, for example one, two, three or four different the peptides of the disclosure. By using a combination of different the peptides of the disclosure the effect may be increased.

The pharmaceutical compositions of the disclosure may also be in the form of a liposome, in which the one or more peptides of the disclosure is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations is can be found in for example U.S. Pat. No. 4,235,871, which is incorporated herein by reference.

The pharmaceutical compositions of the disclosure may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 213 303, which are incorporated herein by reference.

The pharmaceutical compositions of the disclosure may also be formulated with micellar systems formed by surfactants and block copolymers, preferably those containing poly(ethylene oxide) moieties for prolonging bloodstream circulation time. The pharmaceutical compositions of the disclosure may also be in the form of polymer gels, where polymers such as starch, cellulose ethers, cellulose, carboxymethylcellulose, hydroxypropylmethyl cellulose, hydroxyethyl cellulose, ethylhydroxyethyl cellulose, ethyl cellulose, methyl cellulose, propyl cellulose, alginates, chitosan, carageenans, hyaluronic acid and derivatives thereof, polyacrylic acid, polyvinyl imidazole, polysulphonate, polyethylenglycol/polyethylene oxide, polyethylene-oxide/polypropylene oxide copolymers, polyvinylalcohol/polyvinylacetate of different degree of hydrolysis, and polyvinylpyrrolidone are used for thickening of the solution containing the peptide. The polymers may also comprise gelatin or collagen.

Alternatively, the peptides of the disclosure may be dissolved in saline, water, polyethylene glycol, propylene glycol, ethanol or oils (such as safflower oil, corn oil, peanut oil, cottonseed oil or sesame oil), tragacanth gum, and/or various buffers.

The pharmaceutical composition may also include ions and a defined pH for potentiation of action of anti-microbial polypeptides.

The above compositions of the disclosure may be subjected to conventional pharmaceutical operations such as sterilisation and/or may contain conventional adjuvants such as preservatives, stabilisers, wetting agents, emulsifiers, buffers, fillers, etc., e.g., as disclosed elsewhere herein.

It will be appreciated by persons skilled in the art that the pharmaceutical compositions of the disclosure may be administered locally or systemically. Routes of administration include topical (e.g. ophthalmic), ocular, nasal, pulmonary, buccal, parenteral (intravenous, subcutaneous, and intramuscular), oral, vaginal and rectal. Also administration from implants is possible. Suitable preparation forms are, for example granules, powders, tablets, coated tablets, (micro) capsules, suppositories, syrups, emulsions, microemulsions, defined as optically isotropic thermodynamically stable systems consisting of water, oil and surfactant, liquid crystalline phases, defined as systems characterised by long-range order but short-range disorder (examples include lamellar, hexagonal and cubic phases, either water- or oil continuous), or their dispersed counterparts, gels, ointments, dispersions, suspensions, creams, aerosols, droplets or injectable solution in ampoule form and also preparations with protracted release of active compounds, in whose preparation excipients, diluents, adjuvants or carriers are customarily used as described above.

In a particular embodiment, the pharmaceutical composition is suitable for oral administration, parenteral administration or topical administration. For example, the pharmaceutical composition may be suitable for topical administration (e.g. ophthalmic administration, in the form of a spray, lotion, paste or drops etc.).

The pharmaceutical compositions will be administered to a patient in a pharmaceutically effective dose. By “pharmaceutically effective dose” is meant a dose that is sufficient to produce the desired effects in relation to the condition for which it is administered. The exact dose is dependent on the, activity of the compound, manner of administration, nature and severity of the disorder, age and body weight of the patient different doses may be needed. The administration of the dose can be carried out both by single administration in the form of an individual dose unit or else several smaller dose units and also by multiple administrations of subdivided doses at specific intervals.

Compounds of the disclosure may be administered once, twice or thrice per day, especially once or twice per day. A suitable dosage amount may be determined by reference to the severity of the disease and the size of the subject. Typical dosage amounts are in the range 0.01 mg to 100 mg, e.g. 0.1 mg to 10 mg e.g. 0.25 mg to 5 mg per human dose to be delivered once, twice or thrice per day, especially once or twice per day.

The pharmaceutical compositions of the disclosure may be administered alone or in combination with other therapeutic agents, such as anti-cancer agents, anti-Von Hippel-Lindau disease agents, antibiotics, anti-inflammatory, immunosuppressive, vasoactive and/or antiseptic agents (such as anti-bacterial agents, anti-fungicides, anti-viral agents, and anti-parasitic agents). Likewise, the pharmaceutical compositions may also contain anti-inflammatory drugs, such as steroids and macrolactam derivatives.

Such additional therapeutic agents may be incorporated as part of the same pharmaceutical composition or may be administered separately.

In addition to the agents such as peptides and pharmaceutical compositions provided by the disclosure, the disclosure also provides corresponding uses and methods of use of these agents. In all therapeutic uses and methods of the disclosure it will be appreciated that the methods and uses may involve the administration of one, or more than one, for example 2, 3, 4, 5, 6, 7, 8, 9 or 10 different peptides or pharmaceutical compositions according to the disclosure, for example as particular combinations of these agents may have particularly useful therapeutic effects.

The disclosure provides one or more of the peptides or the pharmaceutical compositions according to the disclosure or combination thereof, for use in medicine, for example for use in the treatment of prevention of disease a disorder or a condition as described herein, for example, cancer. The cancer may be a solid cancer or may be a non-solid cancer, for example a blood cancer. Preferably the cancer is a solid cancer.

Preferably the cancer is a cancer:
that experiences a hypoxic environment and requires the typical hypoxia response for maintenance;
that is treatable or preventable by inhibition of dimerization of HIF-1a with HIF1-b and HIF2a with HIF1b and/or inhibits the activity of HIF-1 and HIF-2 and/or HIF-1 or HIF-2 signalling; and/or
in which it is desirable to repress hypoxia induced gene expression.

In some embodiments, the cancer is selected from the group comprising or consisting of:
acute lymphoblastic leukemia (ALL), Acute myeloid leukemia, Adrenocortical carcinoma, AIDS-related cancers, AIDS-related lymphoma, Anal cancer, Appendix cancer, Astrocytoma, childhood cerebellar or cerebral, Basal-cell carcinoma, Bile duct cancer, extrahepatic (see cholangiocarcinoma), Bladder cancer, Bone tumor, osteosarcoma/malignant fibrous histiocytoma, Brainstem glioma, Brain cancer, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Breast cancer, Bronchial adenomas/carcinoids, Burkitt's lymphoma, Carcinoid tumor, childhood, Carcinoid tumor, gastrointestinal, Carcinoma of unknown primary, Cerebellar astrocytoma, Cerebral astrocytoma/malignant glioma, Cervical cancer, Chondrosarcoma, Chronic lymphocytic leukemia, Chronic myelogenous leukemia, Chronic myeloproliferative disorders, Colon cancer, Cutaneous T-cell lymphoma, Desmoplastic small round cell tumor, Endometrial cancer, Ependymoma, Esophageal cancer, Ewing's sarcoma, Extracranial germ cell tumor, Extragonadal germ cell tumor, Extrahepatic bile duct cancer, intraocular melanoma, retinoblastoma, Gallbladder cancer, Gastric (stomach) cancer, Gastrointestinal carcinoid tumor, Gastrointestinal stromal tumor (GIST), Germ cell tumor: extracranial, extragonadal, or ovarian, Gestational trophoblastic tumor, Glioma of the brain stem, Glioma, childhood cerebral astrocytoma, Glioma, childhood visual pathway and hypothalamic, Gastric carcinoid, Hairy cell leukemia, Hepatocellular (liver) cancer, Hodgkin lymphoma, Hypopharyngeal cancer, Hypothalamic and visual pathway glioma, childhood, Intraocular melanoma, Islet cell carcinoma (endocrine pancreas), Kaposi sarcoma, Kidney cancer (renal cell cancer), Laryngeal cancer, Leukaemias, Leukaemia, acute lymphoblastic (also called acute lymphocytic leukaemia), Leukaemia, acute myeloid (also called acute myelogenous leukemia), Leukaemia, chronic lymphocytic (also called chronic lymphocytic leukemia), Leukemia, chronic myelogenous (also called chronic myeloid leukemia), Leukemia, hairy cell, Lip and oral cavity cancer, Liposarcoma, Liver cancer (primary), Lung cancer, non-small cell, Lung cancer, small cell, Lymphomas, Lymphoma, AIDS-related, Lymphoma, Burkitt, Lymphoma, cutaneous T-Cell, Lymphoma, Hodgkin, Lymphomas, Non-Hodgkin, Lymphoma, primary central nervous system, Macroglobulinemia, Waldenstrom, Malignant fibrous histiocytoma of bone/osteosarcoma, Medulloblastoma, Melanoma, Merkel cell cancer, Mesothelioma, Mouth cancer, Multiple endocrine neoplasia syndrome, Multiple myeloma/plasma cell neoplasm, Mycosis fungoides, Myelodysplastic syndromes Myelodysplastic/myeloproliferative diseases, Myelogenous leukemia, chronic Myeloid leukemia, adult acute, Myeloid leukemia, childhood acute, Myeloma, multiple (cancer of the bone-marrow), Myeloproliferative disorders, chronic, Myxoma, Nasal cavity and paranasal sinus cancer, Nasopharyngeal carcinoma, Neuroblastoma, Non-Hodgkin lymphoma, Non-small cell lung cancer, Oligodendroglioma, Oral cancer, Oropharyngeal cancer, Osteosarcoma/malignant fibrous histiocytoma of bone, Ovarian cancer, Ovarian epithelial cancer (surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma, Renal pelvis and ureter, transitional cell cancer, Rhabdomyosarcoma, childhood, Salivary gland cancer, Sarcoma, Ewing family of tumors, Sarcoma, Kaposi, Sarcoma, soft tissue, Sarcoma, uterine, Sezary syndrome, Skin cancer (non-melanoma), Skin cancer (melanoma), Skin carcinoma, Merkel cell, Small cell lung cancer, Small intestine cancer, Soft tissue sarcoma, Squamous cell carcinoma—see skin cancer (non-melanoma), Squamous neck cancer with occult primary, metastatic, Stomach cancer, Supratentorial primitive neuroectodermal tumor, childhood, T-Cell lymphoma, cutaneous, Testicular cancer, Throat cancer, Thymoma, Thymoma and thymic carcinoma, Thyroid cancer, Thyroid cancer, Transitional cell cancer of the renal pelvis and ureter, Trophoblastic tumor, gestational, Unknown primary site, Ureter and renal pelvis, transitional cell cancer, Urethral cancer, Uterine cancer, endometrial, Uterine sarcoma, Vaginal cancer, Visual pathway and hypothalamic glioma, Vulvar cancer, Waldenstrom macroglobulinemia and/or Wilms tumor (kidney cancer).

It will also be clear that any of the peptides according to the disclosure can be formulated with one or more further therapeutic agents, for example one or more further anti-cancer therapeutic agents or one or more further agents for the treatment of Von Hippel-Lindau disease.

It will also be clear that any of the peptides and/or pharmaceutical compositions according to the disclosure can be administered as part of a combination therapy. For example, they can be administered prior to a further therapeutic agent, for example one or more further anti-cancer therapeutic agents or one or more further agents for the treatment of von Hippel-Lindau disease. Alternatively the peptides and/or pharmaceutical compositions can be administered following the administration of a further therapeutic agent, for example one or more further anti-cancer therapeutic agents or one or more further agents for the treatment of von Hippel-Lindau disease.

Alternatively the peptides and/or pharmaceutical compositions according to the disclosure can be administered simultaneously to the administration of a further therapeutic agent, for example one or more further anti-cancer therapeutic agents or one or more further agents for the treatment of von Hippel-Lindau disease. The simultaneous administration may involve the administration of a single composition comprising both the peptides according to the disclosure, and the one or more further therapeutic agents. The simultaneous administration may instead involve the administration of separate compositions, a first composition comprising the peptides according to the disclosure and a second composition comprising the one or more further therapeutic agents.

The disclosure also provides a method for the treatment or prevention of a disease, disorder or condition, wherein the method comprises administration of one or more of peptides and/or pharmaceutical compositions according to the disclosure. Preferences for the disease, peptide and other features are described elsewhere herein.

The disclosure also provides the use of one or more of the peptides and/or pharmaceutical compositions according to the disclosure in a method of manufacture of a medicament for use in medicine, specifically for use in the treatment or prevention of a disease, disorder or condition as described elsewhere herein.

Preferences and options for a given aspect, feature or parameter of the disclosure should, unless the context indicates otherwise, be regarded as having been disclosed in combination with any and all preferences and options for all other aspects, features and parameters of the disclosure.

### Experimental Section

### Abbreviations

Bu butyl
Cys cysteine
Calc calculated
DCM dichloromethane
DIC N,N′-Diisopropylcarbodiimide
DIPEA N,N-Diisopropylethylamine
DMF N,N-dimethylformamide
DMSO dimethyl sulfoxide
DTT dithiothreitol
ESI-MS electrospray ionization mass spectrometry
Equiv equivalent
Fmoc fluorenylmethyloxycarbonyl protecting group
h hour(s)
hLeu homoleucine
HFIP hexafluoroisopropanol
HPLC high performance liquid chromatography
LCMS liquid chromatography mass spectrometry
Leu leucine
[M+H]⁺ protonated molecular ion
[M−H]⁻ deprotonated molecular ion
m/z mass-to-charge ratio
MeCN acetonitrile
min minute(s)
Phe phenylalanine
rt or RT room temperature
SPPS solid-phase peptide synthesis
St S-tert
TFA trifluoroacetic acid
TIS triisopropylsilane
tR retention time
Trt trityl
w weight

### EXAMPLES

### General Procedures

All starting materials and solvents were obtained either from commercial sources or prepared according to the literature citation. Unless otherwise stated all reactions were stirred.

### Reverse Phase Chromatography

Reverse phase chromatography was performed on a Biotage Isolera One system, using linear gradients of solvents A (0.1% TFA/H₂O) and B (0.1% TFA/MeCN). Peptide solutions were loaded into Biotage SNAP Ultra C18 30 g columns dissolved in either a H₂O:MeCN mixture where possible, or DMF. Methods were run at 25 mL/min. The following general methods were used:

### Method A:

| Column volumes | % A | % B |
|---|---|---|
| 1 | 95 | 5 |
| 10 | 5 | 95 |
| 2 | 5 | 95 |

### Method B:

| Column volumes | % A | % B |
|---|---|---|
| 1 | 70 | 30 |
| 10 | 10 | 90 |
| 2 | 10 | 90 |

### Preparative HPLC

Preparative HPLC was performed on an Agilent 1260 Infinity II Manual preparative LC system using linear gradients of solvents A (0.1% TFA/H₂O) and B (0.1% TFA/MeCN).

Peptides were purified by preparative HPLC with a Phenomenex Luna C18 column (5.0 μm particle size, 21.2 250 mm) at 20 mL/min flow rate.

The following general methods were used:

### Method A:

| Time (min) | % A | % B |
|---|---|---|
| 0.00 | 95 | 5 |
| 1.00 | 95 | 5 |
| 15.00 | 5 | 95 |
| 20.00 | 5 | 95 |

### Method B:

| Time (min) | % A | % B |
|---|---|---|
| 0.00 | 80 | 20 |
| 1.00 | 80 | 20 |
| 15.00 | 10 | 90 |
| 20.00 | 10 | 90 |

### Synthesis of the Intermediate Resin-Bound Tripeptide (Int1)

The resin bound tri-peptide (Int1) was synthesized following standard Fmoc chemistry protocol on a Fmoc Rink amide solid support (0.51 mmol/g, 1.32 g, 0.67 mmol). Coupling reactions were carried out in SPPS reaction bubbler adding to the resin a solution of Fmoc-L-Cys (Trt)-OH (3 equiv), DIC (3 equiv), Oxyma Pure (3 equiv) and DIPEA (5 equiv) in 5 mL of DMF, and then bubbling the reaction mixture for 1 h at rt. Upon DMF washing, Fmoc-deprotection was carried out using the standard procedure, bubbling in 20% piperidine in DMF. Upon washing with DMF, DCM and diethyl ether 3 times each. Kaiser test confirmed the Fmoc-deprotection by colouring the resin and solution to blue. Rest of the amino acids, Fmoc-Phe(4-I)—OH and Fmoc-hLeu-OH were coupled and Fmoc-deprotected using the above protocol to afford Intl (1.6 g).

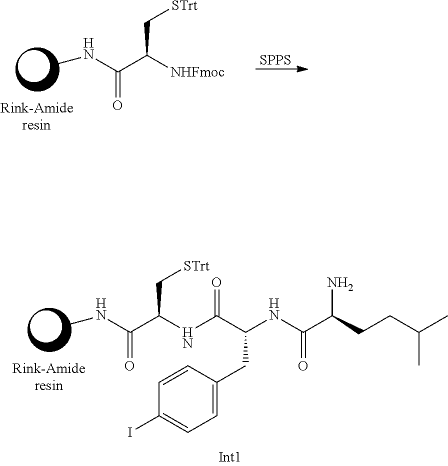

### Synthesis of Example 1

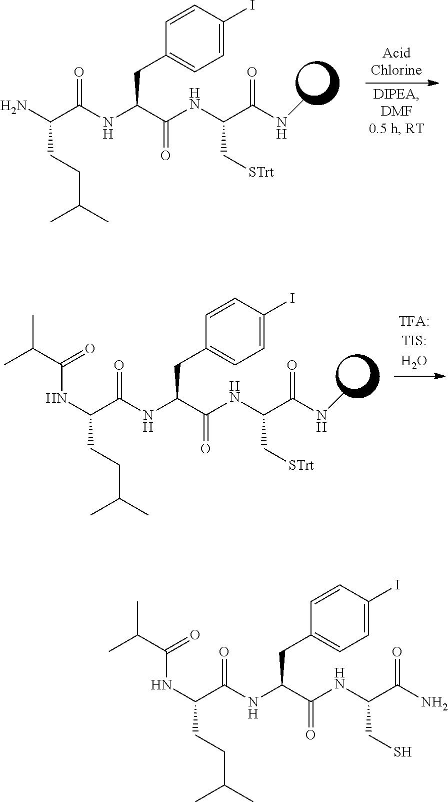

Resin Intl (0.1 g, 0.51 mmol/g) was swollen in DMF and added the corresponding acid chloride/sulfonyl chloride (3 equiv), DIPEA (3 equiv) in 2 mL of DMF to a bubbler and bubbled by nitrogen gas for 30 min. Upon washing resin with DMF, DCM and ether 3 times each, reaction completion was confirmed by Kaiser Test. Entire resin was transferred to a glass vial and added TFA:TIS:H₂O (3 mL:0.15 mL:0.15 mL) and stirred for 2 h at room temperature. Resin was filtered through cotton fitted glass pipette; volatiles were removed under reduced pressure. Crude product was purified by reverse phased-HPLC. After vaccing off the volatile and freeze drying the water white fluffy material Example 1 (6.7 mg, 27%). Analytical HPLC Rt=13.47 min; ESI-MS m/z calcd for molecular formula C₂₃H₃₆IN₄O₄S⁺[M+H]⁺ 591.15, found 591.34 and [2M+H]⁺1181.88.

### Synthesis of Example 2

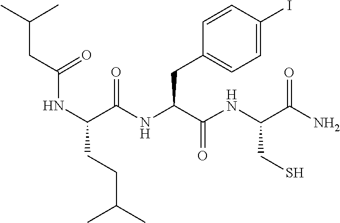 [SMILES: CC(C)CC[C@H](NC(=O)CC(C)C)C(=O)N[C@@H](Cc1ccc(I)cc1)C(=O)N[C@@H](CS)C(N)=O]

Example 2 was synthesized according to the method used for the synthesis of Example 1. Crude product was purified by reverse phased-HPLC. After vac-off the volatile and freeze drying the water white fluffy material Example 2 (7.7 mg, 31%). LCMS Rₜ=2.20 min ESI-MS m/z calcd for molecular formula C₂₄H₃₈IN₄O₄S+[M+H]⁺ 605.17, found 605.38.

### Synthesis of Example 3

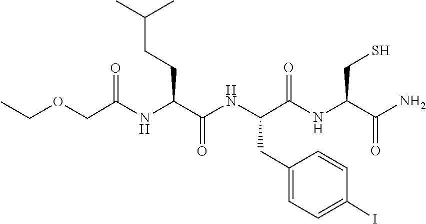 [SMILES: CCOCC(=O)N[C@@H](CCC(C)C)C(=O)N[C@@H](Cc1ccc(I)cc1)C(=O)N[C@@H](CS)C(N)=O]

Example 3 was synthesized according to the protocol used for the synthesis of Example 1. Crude product was purified by reverse phased-HPLC. After vac-off the volatile and freeze drying the water white fluffy material Example 3 (6.5 mg, 27%). Analytical HPLC Rₜ=12.61 min ESI-MS m/z calcd for molecular formula C₂₂H₃₄IN₄O₅S+[M+H]⁺ 593.13, found 593.30.

### Synthesis of Example 4

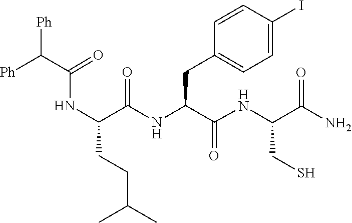 [SMILES: CC(C)CC[C@H](NC(=O)C(c1ccccc1)c1ccccc1)C(=O)N[C@@H](Cc1ccc(I)cc1)C(=O)N[C@@H](CS)C(N)=O]

Example 4 was synthesized according to the protocol used for the synthesis of Example 1. Crude product was purified by reverse phased-HPLC. After vac-off the volatile and freeze drying the water white fluffy material Example 4 (7.6 mg, 26%). Analytical HPLC Rₜ=16.56 min; ESI-MS m/z calcd for molecular formula C₃₃H₄₀IN₄O₄S⁺[M+H]⁺ 714.17, found 714.42.

### Synthesis of Example 5

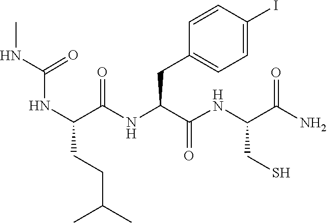 [SMILES: CNC(=O)N[C@@H](CCC(C)C)C(=O)N[C@@H](Cc1ccc(I)cc1)C(=O)N[C@@H](CS)C(N)=O]

Example 5 was synthesized according to the protocol used for the synthesis of Example 1. Crude product was purified by reverse phased-HPLC. After vac-off the volatile and freeze drying the water white fluffy material Example 5 (6.5 mg, 27%). Analytical HPLC Rₜ=12.22 min; ESI-MS m/z calcd for molecular formula C₂₁H₃₃IN₅O₄S⁺[M+H]⁺ 578.13, found 578.31.

### Synthesis of Example 6

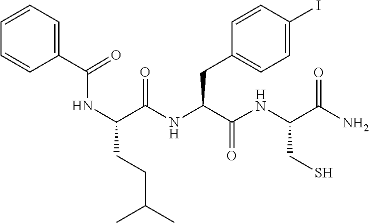 [SMILES: CC(C)CC[C@H](NC(=O)c1ccccc1)C(=O)N[C@@H](Cc1ccc(I)cc1)C(=O)N[C@@H](CS)C(N)=O]

Example 6 was synthesized according to the protocol used for the synthesis of Example 1. Crude product was purified by reverse phased-HPLC. After vac-off the volatile and freeze drying the water white fluffy material Example 6 (8 mg, 31%). LCMS Rₜ=2.22 min; ESI-MS m/z calcd for molecular formula C₂₆H₃₄IN₄O₄S⁺[M+H]⁺ 625.13, found 625.40.

### Synthesis of Example 7

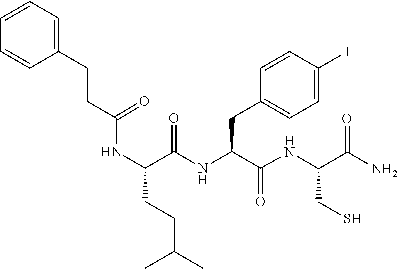 [SMILES: CC(C)CC[C@H](NC(=O)CCc1ccccc1)C(=O)N[C@@H](Cc1ccc(I)cc1)C(=O)N[C@@H](CS)C(N)=O]

Example 7 was synthesized according to the protocol used for the synthesis of Example 1. Crude product was purified by reverse phased-HPLC. After vac-off the volatile and freeze drying the water white fluffy material Example 7 (3 mg, 11%). Analytical HPLC Rₜ=12.86 min ESI-MS m/z calcd for molecular formula C₂₈H₃₈IN₄O₄S⁺[M+H]⁺ 653.17, found 653.52.

### Synthesis of Example 8

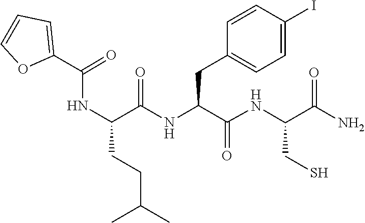 [SMILES: CC(C)CC[C@H](NC(=O)c1ccco1)C(=O)N[C@@H](Cc1ccc(I)cc1)C(=O)N[C@@H](CS)C(N)=O]

Example 8 was synthesized according to the protocol used for the synthesis of Example 1. Crude product was purified by reverse phased-HPLC. After vac-off the volatile and freeze drying the water white fluffy material Example 8 (6 mg, 24%). Analytical HPLC Rₜ=13.61 min; ESI-MS m/z calcd for molecular formula C₂₄H₃₂IN₄O₅S⁺[M+H]⁺ 615.11, found 615.30.

### Synthesis of Example 9

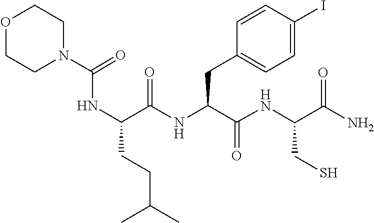 [SMILES: CC(C)CC[C@H](NC(=O)N1CCOCC1)C(=O)N[C@@H](Cc1ccc(I)cc1)C(=O)N[C@@H](CS)C(N)=O]

Example 9 was synthesized according to the protocol used for the synthesis of Example 1. Crude product was purified by reverse phased-HPLC. After vac-off the volatile and freeze drying the water white fluffy material Example 9 (4 mg, 15%). Analytical HPLC Rₜ=12.63 min; ESI-MS m/z calcd for molecular formula C₂₄H₃₇IN₅O₅S⁺[M+H]⁺ 634.16, found 634.45.

### Synthesis of Example 10

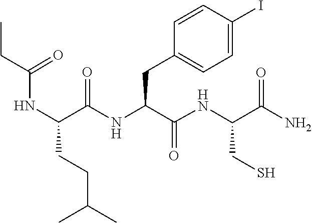 [SMILES: CCC(=O)N[C@@H](CCC(C)C)C(=O)N[C@@H](Cc1ccc(I)cc1)C(=O)N[C@@H](CS)C(N)=O]

Example 10 was synthesized according to the protocol used for the synthesis of Example 1. Crude product was purified by reverse phased-HPLC. After vac-off the volatile and freeze drying the water white fluffy material Example 10 (4 mg, 17%). Analytical HPLC Rₜ=12.90 min; ESI-MS m/z calcd for molecular formula C²²H³⁴IN⁴O⁴S⁺[M+H]⁺ 577.13, found 577.41.

### Synthesis of Example 11

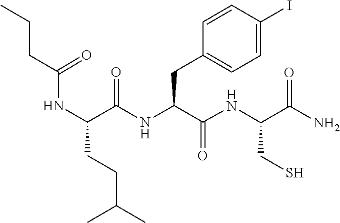 [SMILES: CCCC(=O)N[C@@H](CCC(C)C)C(=O)N[C@@H](Cc1ccc(I)cc1)C(=O)N[C@@H](CS)C(N)=O]

Example 11 was synthesized according to the protocol used for the synthesis of Example 1. Crude product was purified by reverse phased-HPLC. After vac-off the volatile and freeze drying the water white fluffy material Example 11 (5.2 mg, 21%). Analytical HPLC Rₜ=13.57 min; ESI-MS m/z calcd for molecular formula C₂₃H₃₆IN₄O₄S⁺[M+H]⁺ 591.15, found 591.42.

### Synthesis of Example 12

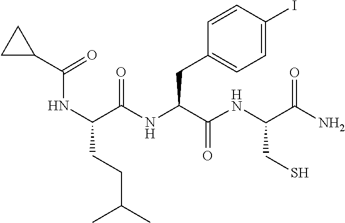 [SMILES: CC(C)CC[C@H](NC(=O)C1CC1)C(=O)N[C@@H](Cc1ccc(I)cc1)C(=O)N[C@@H](CS)C(N)=O]

Example 12 was synthesized according to the protocol used for the synthesis of Example 1. Crude product was purified by reverse phased-HPLC. After vac-off the volatile and freeze drying the water white fluffy material Example 12 (4.9 mg, 20%). LCMS Rₜ=2.04 min; ESI-MS m/z calcd for molecular formula C₂₃H₃₄IN₄O₄S⁺[M+H]⁺ 589.13, found 589.43.

### Synthesis of Example 13

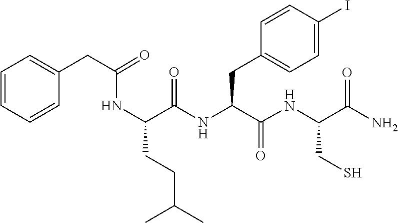 [SMILES: CC(C)CC[C@H](NC(=O)Cc1ccccc1)C(=O)N[C@@H](Cc1ccc(I)cc1)C(=O)N[C@@H](CS)C(N)=O]

Example 13 was synthesized according to the protocol used for the synthesis of Example 1. Crude product was purified by reverse phased-HPLC. After vac-off the volatile and freeze drying the water white fluffy material Example 13 (9.5 mg, 36%). LCMS Rₜ=2.22 min; ESI-MS m/z calcd for molecular formula C₂₇H₃₆IN₄O₄S⁺[M+H]⁺ 639.15, found 639.41.

### Synthesis of Example 14

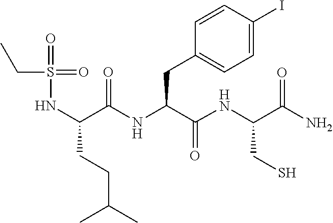 [SMILES: CCS(=O)(=O)N[C@@H](CCC(C)C)C(=O)N[C@@H](Cc1ccc(I)cc1)C(=O)N[C@@H](CS)C(N)=O]

Example 14 was synthesized according to the protocol used for the synthesis of Example 1. Crude product was purified by reverse phased-HPLC. After vac-off the volatile and freeze drying the water white fluffy material Example 14 (2.5 mg, 10%). LCMS Rₜ=1.97 min; ESI-MS m/z calcd for molecular formula C₂₁H₃₄IN₄O₅S₂⁺[M+Na]⁺635.08, found 613.33.

### Synthesis of Example 15

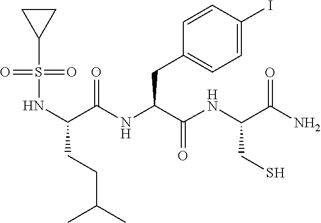 [SMILES: CC(C)CC[C@H](NS(=O)(=O)C1CC1)C(=O)N[C@@H](Cc1ccc(I)cc1)C(=O)N[C@@H](CS)C(N)=O]

Example 15 was synthesized according to the protocol used for the synthesis of Example 1. Crude product was purified by reverse phased-HPLC. After vac-off the volatile and freeze drying the water white fluffy material Example 15 (14 mg, 55%). LCMS Rₜ=2.01 min; ESI-MS m/z calcd for molecular formula C₂₂H₃₄IN₄O₅S₂⁺[M+H]⁺ 625.10, found 625.30.

### Synthesis of Example 16

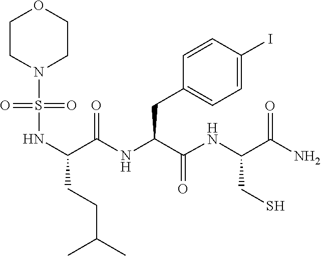 [SMILES: CC(C)CC[C@H](NS(=O)(=O)N1CCOCC1)C(=O)N[C@@H](Cc1ccc(I)cc1)C(=O)N[C@@H](CS)C(N)=O]

Example 16 was synthesized according to the protocol used for the synthesis of Example 1. Crude product was purified by reverse phased-HPLC. After vac-off the volatile and freeze drying the water white fluffy material Example 16 (11 mg, 38%). Analytical HPLC Rₜ=13.09 min; ESI-MS m/z calcd for molecular formula C₂₃H₃₇IN₅O₆S₂⁺[M+H]⁺ 670.12, found 670.32.

### Synthesis of Example 17

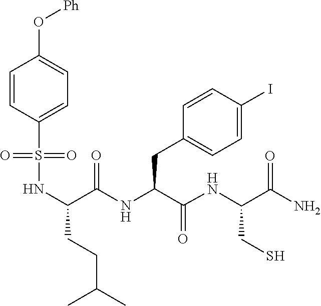 [SMILES: CC(C)CC[C@H](NS(=O)(=O)c1ccc(Oc2ccccc2)cc1)C(=O)N[C@@H](Cc1ccc(I)cc1)C(=O)N[C@@H](CS)C(N)=O]

Example 17 was synthesized according to the protocol used for the synthesis of Example 1. Crude product was purified by reverse phased-HPLC. After vac-off the volatile and freeze drying the water white fluffy material Example 17 (22 mg, 71%). Analytical HPLC Rₜ=16.60 min; ESI-MS m/z calcd for molecular formula C₃₁H₃₈IN₄O₆S₂⁺[M+H]⁺ 753.13, found 753.36.

### Synthesis of Example 18

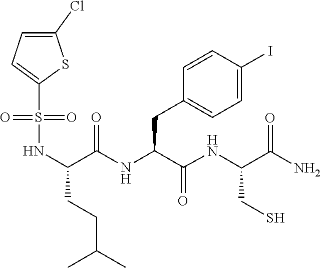 [SMILES: CC(C)CC[C@H](NS(=O)(=O)c1ccc(Cl)s1)C(=O)N[C@@H](Cc1ccc(I)cc1)C(=O)N[C@@H](CS)C(N)=O]

Example 18 was synthesized according to the protocol used for the synthesis of Example 1. Crude product was purified by reverse phased-HPLC. After vac-off the volatile and freeze drying the water white fluffy material Example 18 (22 mg, 77%). Analytical HPLC Rₜ=15.39 min; ESI-MS m/z calcd for molecular formula C₂₃H₃₁ClIN₄O₅S₃⁺[M+Na]⁺723.00, found 723.26.

### Synthesis of Example 19

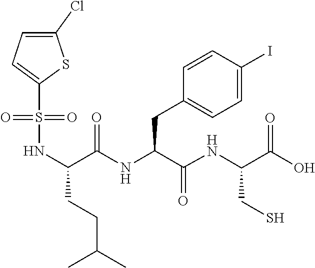 [SMILES: CC(C)CC[C@H](NS(=O)(=O)c1ccc(Cl)s1)C(=O)N[C@@H](Cc1ccc(I)cc1)C(=O)N[C@@H](CS)C(=O)O]

Example 19 was synthesized according to the protocol used for the synthesis of Example 1. Crude product was purified by reverse phased-HPLC. After vac-off the volatile and freeze drying the water white fluffy material Example 19 (17 mg, 59%). LCMS Rₜ=2.61 min; ESI-MS m/z calcd for molecular formula C₂₃H₃₀ClIN₃O₆S₃⁺[M+Na]⁺702.00, found 702.3.

### Synthesis of Example 20

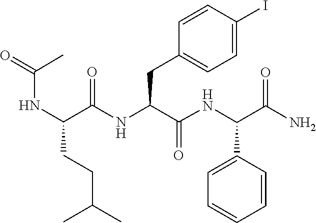 [SMILES: CC(=O)N[C@@H](CCC(C)C)C(=O)N[C@@H](Cc1ccc(I)cc1)C(=O)N[C@H](C(N)=O)c1ccccc1]

Example 20 was synthesized according to the protocol used for the synthesis of Example 1. Crude product was purified by reverse phased-HPLC. After vac-off the volatile and freeze drying the water white fluffy material Example 20 (6 mg, 25%). Analytical HPLC Rₜ=12.86 min; ESI-MS m/z calcd for molecular formula C₂₆H₃₄IN₄O₄⁺[M+H]⁺ 593.16, found 593.30.

### Synthesis of Example 21

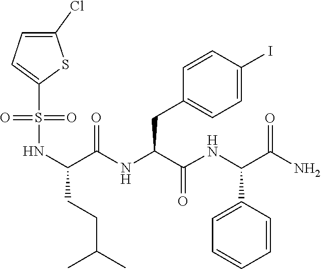 [SMILES: CC(C)CC[C@H](NS(=O)(=O)c1ccc(Cl)s1)C(=O)N[C@@H](Cc1ccc(I)cc1)C(=O)N[C@H](C(N)=O)c1ccccc1]

Example 21 was synthesized according to the protocol used for the synthesis of Example 1. Crude product was purified by reverse phased-HPLC. After vac-off the volatile and freeze drying the water white fluffy material Example 21 (8 mg, 27%). Analytical HPLC Rₜ=16.16 min; ESI-MS m/z calcd for molecular formula C₂₈H₃₃ClIN₄O₅S₂⁺[M+Na]⁺731.06, found 723.40.

### Synthesis of Example 22

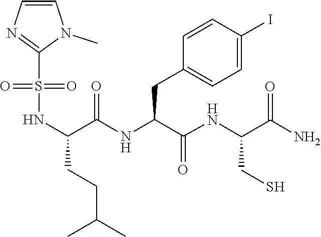 [SMILES: CC(C)CC[C@H](NS(=O)(=O)c1nccn1C)C(=O)N[C@@H](Cc1ccc(I)cc1)C(=O)N[C@@H](CS)C(N)=O]

Example 22 was synthesized according to the protocol used for the synthesis of Example 1. Crude product was purified by reverse phased-HPLC. After vac-off the volatile and freeze drying the water white fluffy material Example 22 (15 mg, 55%). Analytical HPLC Rₜ=12.45 min; ESI-MS m/z calcd for molecular formula C₂₃H₃₄IN₆O₅S₂⁺[M+H]⁺ 665.11, found 665.43.

### Synthesis of Example 23

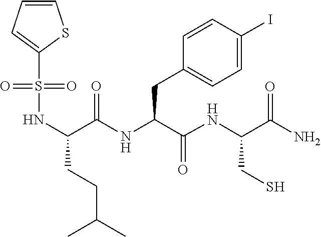 [SMILES: CC(C)CC[C@H](NS(=O)(=O)c1cccs1)C(=O)N[C@@H](Cc1ccc(I)cc1)C(=O)N[C@@H](CS)C(N)=O]

Example 23 was synthesized according to the protocol used for the synthesis of Example 1. Crude product was purified by reverse phased-HPLC. After vac-off the volatile and freeze drying the water white fluffy material Example 23 (22 mg, 82%). Analytical HPLC Rₜ=14.10 min; ESI-MS m/z calcd for molecular formula C₂₃H₃₂IN₄O₅S₃⁺[M+H]⁺ 667.06, found 667.25.

### Synthesis of Example 24

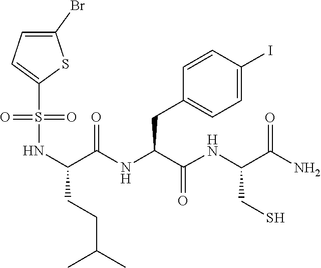 [SMILES: CC(C)CC[C@H](NS(=O)(=O)c1ccc(Br)s1)C(=O)N[C@@H](Cc1ccc(I)cc1)C(=O)N[C@@H](CS)C(N)=O]

Example 24 was synthesized according to the protocol used for the synthesis of Example 1. Crude product was purified by reverse phased-HPLC. After vac-off the volatile and freeze drying the water white fluffy material Example 24 (22 mg, 72%). Analytical HPLC Rₜ=13.25 min; ESI-MS m/z calcd for molecular formula C₂₃H₃₀BrIN₄NaO₅S₃⁺[M+Na]⁺768.49, found 769.11.

### Synthesis of Example 25

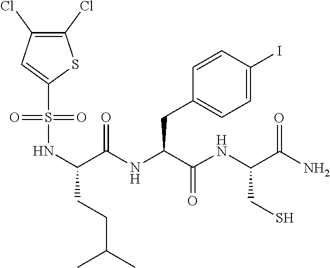 [SMILES: CC(C)CC[C@H](NS(=O)(=O)c1cc(Cl)c(Cl)s1)C(=O)N[C@@H](Cc1ccc(I)cc1)C(=O)N[C@@H](CS)C(N)=O]

Example 25 was synthesized according to the protocol used for the synthesis of Example 1. Crude product was purified by reverse phased-HPLC. After vac-off the volatile and freeze drying the water white fluffy material Example 25 (9.1 mg, 35%). Analytical HPLC Rₜ=16.37 min; ESI-MS m/z calcd for molecular formula C₂₃H₃₀Cl₂IN₄O₅S₃⁺[M+H]⁺ 758.48, found 758.90.

### Synthesis of Example 26

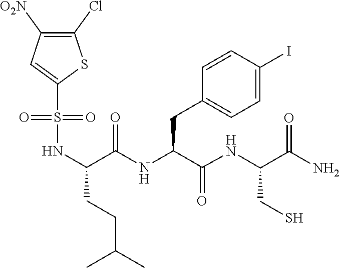 [SMILES: CC(C)CC[C@H](NS(=O)(=O)c1cc([N+](=O)[O-])c(Cl)s1)C(=O)N[C@@H](Cc1ccc(I)cc1)C(=O)N[C@@H](CS)C(N)=O]

Example 26 was synthesized according to the protocol used for the synthesis of Example 1. Crude product was purified by reverse phased-HPLC. After vac-off the volatile and freeze drying the water white fluffy material Example 26 (5.8 mg, 19%). Analytical HPLC Rₜ=15.21 min; ESI-MS m/z calcd for molecular formula C₂₃H₃₀ClIN₅O₇S₃⁺[M+H]⁺ 746.00, found 746.12.

### Synthesis of Example 27

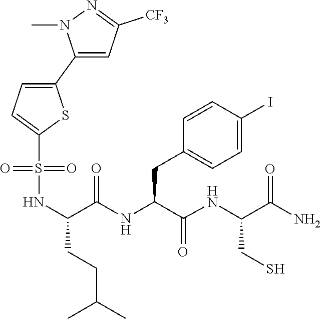 [SMILES: CC(C)CC[C@H](NS(=O)(=O)c1ccc(-c2cc(C(F)(F)F)nn2C)s1)C(=O)N[C@@H](Cc1ccc(I)cc1)C(=O)N[C@@H](CS)C(N)=O]

Example 27 was synthesized according to the protocol used for the synthesis of Example 1. Crude product was purified by reverse phased-HPLC. After vac-off the volatile and freeze drying the water white fluffy material Example 27 (5 mg, 15%). Analytical HPLC Rₜ=17.05 min; ESI-MS m/z calcd for molecular formula C₂₈H₃₄F₃IN₆NaO₅S₃⁺[M+Na]⁺837.06, found 837.35.

### Synthesis of Example 28

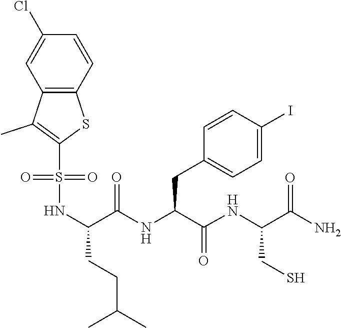 [SMILES: Cc1c(S(=O)(=O)N[C@@H](CCC(C)C)C(=O)N[C@@H](Cc2ccc(I)cc2)C(=O)N[C@@H](CS)C(N)=O)sc2ccc(Cl)cc12]

Example 28 was synthesized according to the protocol used for the synthesis of Example 1. Crude product was purified by reverse phased-HPLC. After vac-off the volatile and freeze drying the water white fluffy material Example 28 (9 mg, 29%). SH Analytical HPLC Rₜ=16.08 min; ESI-MS m/z calcd for molecular formula C₂₈H₃₅ClIN₄O₅S₃⁺[M+H]⁺ 765.05, found 765.16.

### Synthesis of Example 29

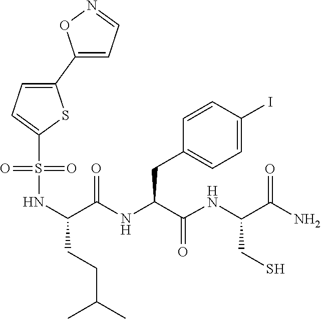 [SMILES: CC(C)CC[C@H](NS(=O)(=O)c1ccc(-c2ccno2)s1)C(=O)N[C@@H](Cc1ccc(I)cc1)C(=O)N[C@@H](CS)C(N)=O]

Example 29 was synthesized according to the protocol used for the synthesis of Example 1. Crude product was purified by reverse phased-HPLC. After vac-off the volatile and freeze drying the water white fluffy material Example 29 (7 mg, 23%). Analytical HPLC Rₜ=14.41 min; ESI-MS m/z calcd for molecular formula C₂₆H₃₃IN₅O₆S₃⁺[M+H]⁺ 734.06, found 734.21.

### Synthesis of Example 30

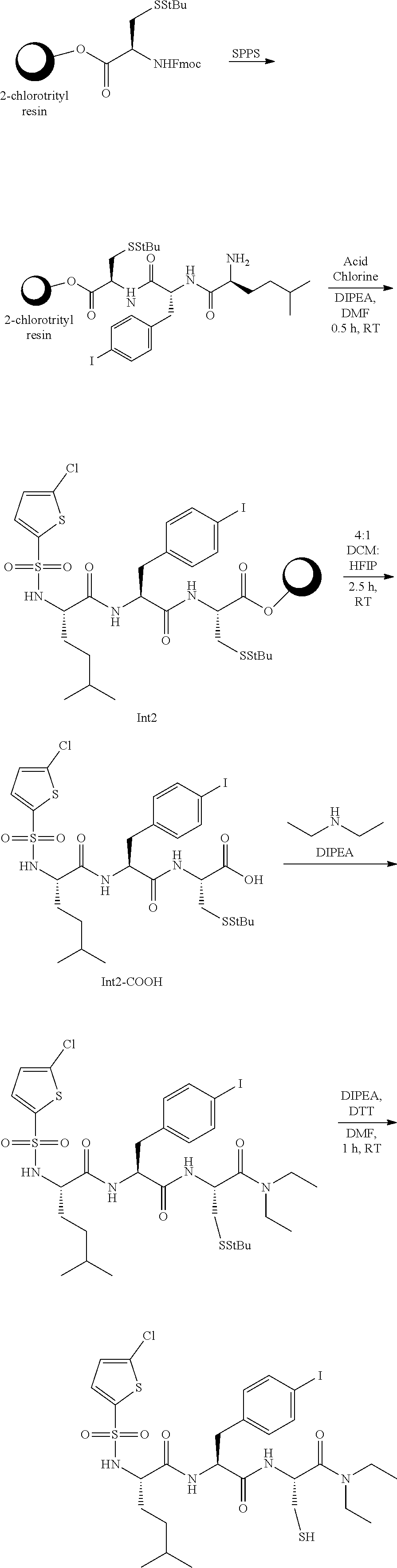

The tri-peptide (Example 30) was synthesized following standard Fmoc chemistry protocol on a Fmoc 2-Chlorotrityl chloride resin solid support (1.47 mmol/g, 2.0 g, 0.2.14 mmol). First loading of Fmoc-L-Cys (StBu)-OH carried out on the resin using DIPEA (5 equiv) in 5 mL of DMF, and then bubbling the reaction mixture for 1 h at RT. Upon DMF washing, Fmoc-deprotection was carried out using the standard procedure, bubbling in 20% piperidine in DMF. Upon washing with DMF, DCM and diethyl ether 3 times each. Kaiser test confirmed the Fmoc-deprotection by colouring the resin and solution to blue. Rest of the amino acids, Fmoc-Phe (4-I)—OH and Fmoc-hLeu-OH were coupled and Fmoc-deprotected using the standard SPPS protocol to afford the resin bound tri-peptide intermediate, Int2 (2.48 g).

The resin bound tri-peptide intermediate was swollen in DMF and added the corresponding sulfonyl chloride (3 equiv), DIPEA (5 equiv) in 2 mL of DMF to a bubbler and bubbled by nitrogen gas for 30 min. Upon washing resin with DMF, DCM and ether 3 times each, reaction completion was confirmed by Kaiser Test. Entire resin (2.48 g) was transferred to a flask and added DCM: HFIP (16 mL: 4 mL) and stirred for 2.5 h at room temperature. Resin was filtered through cotton fitted glass pipette; volatiles were removed under reduced pressure. Crude product was washed with cold ether twice and applied high vacuum overnight to afford white solid Int2-COOH (0.86 g, 51% yield).

Int2-COOH (40 mg, 50 mmol) was stirred with diethylamine (2 equiv.), EDC hydrochloride (2 equiv), HOBt (2 equiv), DIPEA (4 equiv.) in DMF (2 mL) for 1 h at RT (LCMS control). Added 5 mL of water and ethylacetate (2×5 mL). Combined organic layers were dried over MgSO4. Volatiles were removed under reduced pressure. Crude product was directly taken for final reaction without further purification. To the crude product was added excess DIPEA in 1.8 mL DMF and stirred 5 mins and added DTT, stirred 45 min (LCMS control). The reaction mixture was filtered and purified through reverse phased-HPLC. After vaccing off the volatile and freeze drying the water white fluffy material Example 30 (8.5 mg, 22%). Analytical HPLC Rₜ=16.43 min; ESI-MS m/z calcd for molecular formula [M+H]⁺ 757.08, found 757.32.

### Synthesis of Example 31

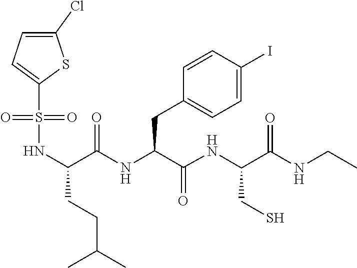 [SMILES: CCNC(=O)[C@H](CS)NC(=O)[C@H](Cc1ccc(I)cc1)NC(=O)[C@H](CCC(C)C)NS(=O)(=O)c1ccc(Cl)s1]

Example 31 was synthesized from Int2 based on the method used for the synthesis of Example 30, on same scale. Crude product was purified by reverse phased-HPLC. After vac-off the volatile and freeze drying the water white fluffy material Example 31 (21 mg, 58%). Analytical HPLC Rₜ=16.43 min; ESI-MS m/z calcd for molecular formula C₂₅H₃₅ClIN₄O₅S₃⁺[M+H]⁺ 729.05, found 729.32.

### Synthesis of Example 32

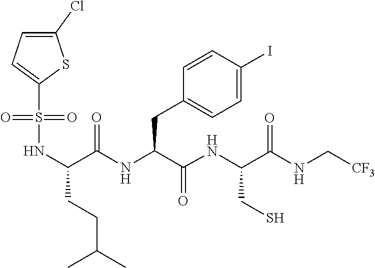 [SMILES: CC(C)CC[C@H](NS(=O)(=O)c1ccc(Cl)s1)C(=O)N[C@@H](Cc1ccc(I)cc1)C(=O)N[C@@H](CS)C(=O)NCC(F)(F)F]

Example 32 was synthesized from Int2 based on the method used for the synthesis of Example 30, on same scale. Crude product was purified by reverse phased-HPLC. After vac-off the volatile and freeze drying the water white fluffy material Example 32 (6.5 mg, 17%). Analytical HPLC Rₜ=17.38 min; ESI-MS m/z calcd for molecular formula C₂₅H₃₂ClF₃IN₄O₅S₃⁺[M+H]⁺ 783.02, found 783.28.

### Synthesis of Example 33

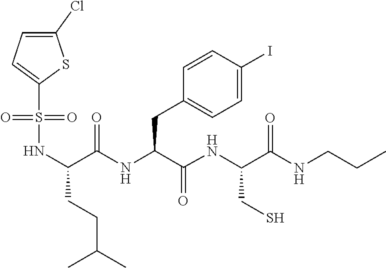 [SMILES: CCCNC(=O)[C@H](CS)NC(=O)[C@H](Cc1ccc(I)cc1)NC(=O)[C@H](CCC(C)C)NS(=O)(=O)c1ccc(Cl)s1]

Example 33 was synthesized from Int2 based on the method used for the synthesis of Example 30, on same scale. Crude product was purified by reverse phased-HPLC. After vac-off the volatile and freeze drying the water white fluffy material Example 33 (6 mg, 16%). Analytical HPLC Rₜ=17.07 min; ESI-MS m/z calcd for molecular formula C₂₆H₃₇ClIN₄O₅S₃⁺[M+H]⁺ 743.07, found 739.31.

### Synthesis of Example 34

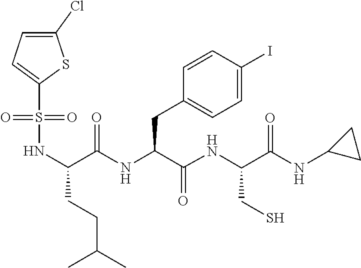 [SMILES: CC(C)CC[C@H](NS(=O)(=O)c1ccc(Cl)s1)C(=O)N[C@@H](Cc1ccc(I)cc1)C(=O)N[C@@H](CS)C(=O)NC1CC1]

Example 34 was synthesized from Int2 based on the method used for the synthesis of Example 30, on same scale. Crude product was purified by reverse phased-HPLC. After vac-off the volatile and freeze drying the water white fluffy material Example 34 (6 mg, 16%). Analytical HPLC Rₜ=16.53 min; ESI-MS m/z calcd for molecular formula C₂₆H₃₅ClIN₄O₅S₃⁺[M+H]⁺ 741.05, found 741.26.

### Synthesis of Example 35

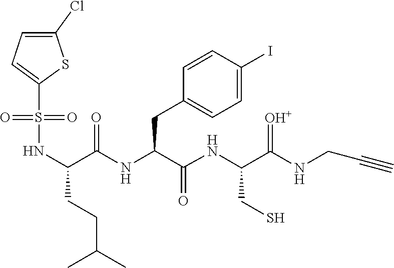 [SMILES: C#CCNC(=[OH+])[C@H](CS)NC(=O)[C@H](Cc1ccc(I)cc1)NC(=O)[C@H](CCC(C)C)NS(=O)(=O)c1ccc(Cl)s1]

Example 35 was synthesized from Int2 based on the method used for the synthesis of Example 30, on same scale. Crude product was purified by reverse phased-HPLC. After vac-off the volatile and freeze drying the water white fluffy material Example 35 (17 mg, 46%). Analytical HPLC Rₜ=16.58 min; ESI-MS m/z calcd for molecular formula C₂₆H₃₃ClIN₄O₅S₃⁺[M+H]⁺ 739.03, found 739.27.

### Synthesis of Example 36

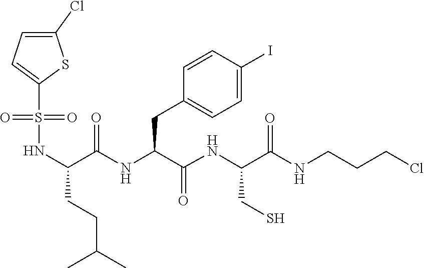 [SMILES: CC(C)CC[C@H](NS(=O)(=O)c1ccc(Cl)s1)C(=O)N[C@@H](Cc1ccc(I)cc1)C(=O)N[C@@H](CS)C(=O)NCCCCl]

Example 36 was synthesized from Int2 based on the method used for the synthesis of Example 30, on same scale. Crude product was purified by reverse phased-HPLC. After vac-off the volatile and freeze drying the water white fluffy material Example 36 (19 mg, 49%). Analytical HPLC Rₜ=18.35 min; ESI-MS m/z calcd for molecular formula C₂₆H₃₆Cl₂IN₄O₅S₃⁺[M+H]⁺ 777.01, found 799.28.

### Synthesis of Example 37

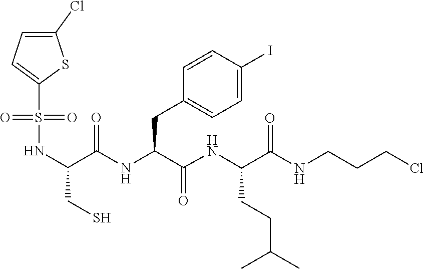 [SMILES: CC(C)CC[C@H](NC(=O)[C@H](Cc1ccc(I)cc1)NC(=O)[C@H](CS)NS(=O)(=O)c1ccc(Cl)s1)C(=O)NCCCCl]

Example 37 was synthesized from Int2 based on the method used for the synthesis of Example 30, on same scale. Crude product was purified by reverse phased-HPLC. After vac-off the volatile and freeze drying the water white fluffy material Example 37 (15 mg, 39%). Analytical HPLC Rₜ=18.35 min; ESI-MS m/z calcd for molecular formula C₂₆H₃₆Cl₂IN₄O₅S₃⁺[M+H]⁺ 777.01, found 777.02.

### Synthesis of Example 38

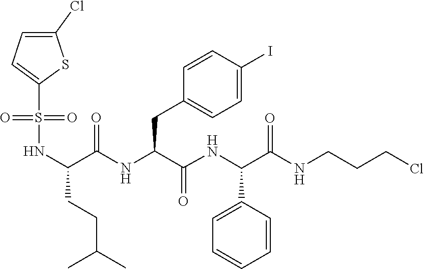 [SMILES: CC(C)CC[C@H](NS(=O)(=O)c1ccc(Cl)s1)C(=O)N[C@@H](Cc1ccc(I)cc1)C(=O)N[C@H](C(=O)NCCCCl)c1ccccc1]

Example 38 was synthesized from Int2 based on the method used for the synthesis of Example 30, on same scale. Crude product was purified by reverse phased-HPLC. After vac-off the volatile and freeze drying the water white fluffy material Example 38 (8 g, 20%). Analytical HPLC Rₜ=17.92 min; ESI-MS m/z calcd for molecular formula C₃₁H₃₈Cl₂IN₄O₅S₂⁺[M+H]⁺ 807.7, found 807.3.

### Synthesis of Example 39

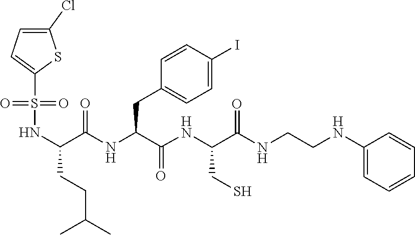 [SMILES: CC(C)CC[C@H](NS(=O)(=O)c1ccc(Cl)s1)C(=O)N[C@@H](Cc1ccc(I)cc1)C(=O)N[C@@H](CS)C(=O)NCCNc1ccccc1]

Example 39 was synthesized from Int2 based on the method used for the synthesis of Example 30, on same scale. Crude product was purified by reverse phased-HPLC. After vac-off the volatile and freeze drying the water white fluffy material Example 39 (5.4 mg, 13%). Analytical HPLC Rₜ=16.08 min; ESI-MS m/z calcd for molecular formula C₃₁H₄₀ClIN₅O₅S₃⁺[M+H]⁺ 820.09, found 820.37.

### Synthesis of Example 40

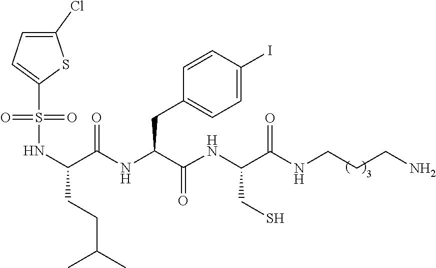

Example 40 was synthesized from Int2 based on the method used for the synthesis of Example 30, on same scale. Crude product was purified by reverse phased-HPLC. After vac-off the volatile and freeze drying the water white fluffy material Example 40 (12 mg, 31%). LCMS Rₜ=2.68 min; ESI-MS m/z calcd for molecular formula C₂₈H₄₂ClIN₃O₃S₃⁺[M+H]⁺ 786.11, found 786.35.

### Synthesis of Example 41

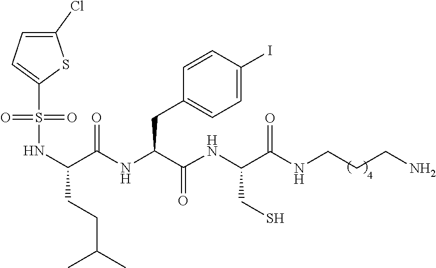

Example 41 was synthesized from Int2 based on the method used for the synthesis of Example 30, on same scale. Crude product was purified by reverse phased-HPLC. After vac-off the volatile and freeze drying the water white fluffy material Example 41 (5.2 mg, 13%). LCMS Rₜ=2.72 min; ESI-MS m/z calcd for molecular formula C₂₉H₄₄ClIN₅O₅S₃⁺[M+H]⁺ 800.12, found 800.37.

### Synthesis of Example 42

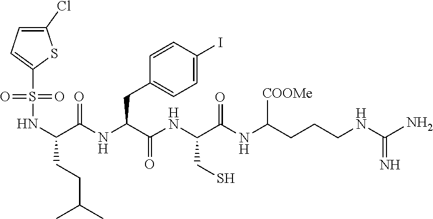 [SMILES: COC(=O)C(CCCNC(=N)N)NC(=O)[C@H](CS)NC(=O)[C@H](Cc1ccc(I)cc1)NC(=O)[C@H](CCC(C)C)NS(=O)(=O)c1ccc(Cl)s1]

Example 42 was synthesized from Int2 based on the method used for the synthesis of Example 30, on same scale. Crude product was purified by reverse phased-HPLC. After vac-off the volatile and freeze drying the water white fluffy material Example 42 (8.5 mg, 20%). LCMS Rₜ=2.01 min; ESI-MS m/z calcd for molecular formula C₃₀H₄₄ClIN₇O₇S₃⁺[M+H]⁺ 872.12, found 872.38.

### Synthesis of Example 43

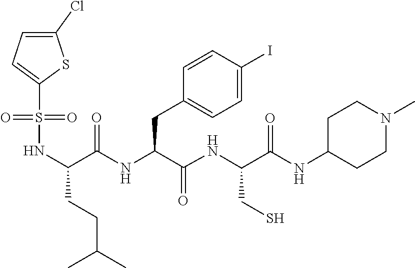 [SMILES: CC(C)CC[C@H](NS(=O)(=O)c1ccc(Cl)s1)C(=O)N[C@@H](Cc1ccc(I)cc1)C(=O)N[C@@H](CS)C(=O)NC1CCN(C)CC1]

Example 43 was synthesized from Int2 based on the method used for the synthesis of Example 30, on same scale. Crude product was purified by reverse phased-HPLC. After vac-off the volatile and freeze drying the water white fluffy material Example 43 (10 mg, 25%). Analytical HPLC Rₜ=13.33 min [Gradient:]; ESI-MS m/z calcd for molecular formula C₂₉H₄₂ClIN₅O₅S₃⁺[M+H]⁺ 798.11, found 798.39.

### Synthesis of Example 44

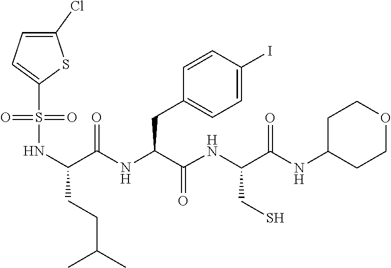 [SMILES: CC(C)CC[C@H](NS(=O)(=O)c1ccc(Cl)s1)C(=O)N[C@@H](Cc1ccc(I)cc1)C(=O)N[C@@H](CS)C(=O)NC1CCOCC1]

Example 44 was synthesized from Int2 based on the method used for the synthesis of Example 30, on same scale. Crude product was purified by reverse phased-HPLC. After vac-off the volatile and freeze drying the water white fluffy material Example 44 (10 mg, 26%). Analytical HPLC Rₜ=16.03 min; ESI-MS m/z calcd for molecular formula C₂₈H₃₉ClIN₄O₆S₃⁺[M+H]⁺ 785.08, found 785.24.

### Synthesis of Example 45

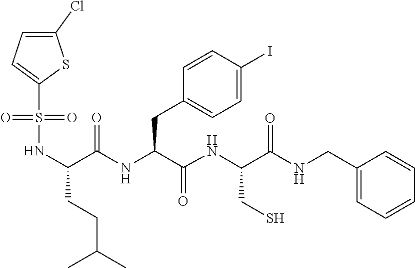 [SMILES: CC(C)CC[C@H](NS(=O)(=O)c1ccc(Cl)s1)C(=O)N[C@@H](Cc1ccc(I)cc1)C(=O)N[C@@H](CS)C(=O)NCc1ccccc1]

Example 45 was synthesized from Int2 based on the method used for the synthesis of Example 30, on same scale. Crude product was purified by reverse phased-HPLC. After vac-off the volatile and freeze drying the water white fluffy material Example 45 (9.5 mg, 24%). Analytical HPLC Rₜ=17.92 min; ESI-MS m/z calcd for molecular formula C₃₀H₃₇ClIN₄O₅S₃⁺[M+H]⁺ 791.07, found 791.20.

### Synthesis of Example 46

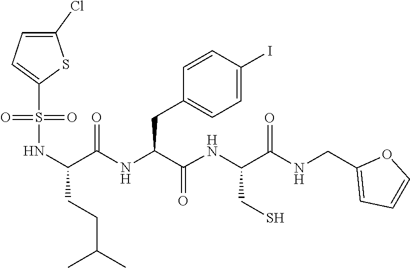 [SMILES: CC(C)CC[C@H](NS(=O)(=O)c1ccc(Cl)s1)C(=O)N[C@@H](Cc1ccc(I)cc1)C(=O)N[C@@H](CS)C(=O)NCc1ccco1]

Example 46 was synthesized from Int2 based on the method used for the synthesis of Example 30, on same scale. Crude product was purified by reverse phased-HPLC. After vac-off the volatile and freeze drying the water white fluffy material Example 46 (11 mg, 28%). Analytical HPLC Rₜ=17.26 min; ESI-MS m/z calcd for molecular formula C₂₈H₃₅ClIN₄O₆S₃⁺[M+Na]⁺781.04, found 781.22.

### Synthesis of Example 47

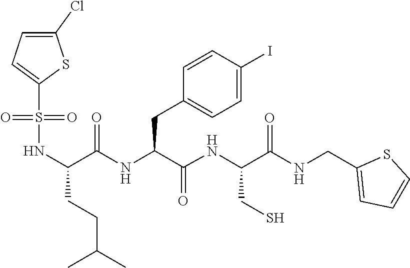 [SMILES: CC(C)CC[C@H](NS(=O)(=O)c1ccc(Cl)s1)C(=O)N[C@@H](Cc1ccc(I)cc1)C(=O)N[C@@H](CS)C(=O)NCc1cccs1]

Example 47 was synthesized from Int2 based on the method used for the synthesis of Example 30, on same scale. Crude product was purified by reverse phased-HPLC. After vac-off the volatile and freeze drying the water white fluffy material Example 47 (9 mg, 23%). Analytical HPLC Rₜ=17.67 min; ESI-MS m/z calcd for molecular formula C₂₈H₃₅ClIN₄O₆S₄⁺[M+H]⁺ 797.02, found 797.18.

### Synthesis of Example 48

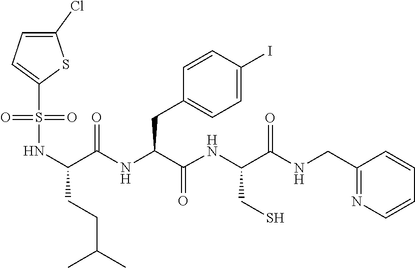 [SMILES: CC(C)CC[C@H](NS(=O)(=O)c1ccc(Cl)s1)C(=O)N[C@@H](Cc1ccc(I)cc1)C(=O)N[C@@H](CS)C(=O)NCc1ccccn1]

Example 48 was synthesized from Int2 based on the method used for the synthesis of Example 30, on same scale. Crude product was purified by reverse phased-HPLC. After vac-off the volatile and freeze drying the water white fluffy material Example 48 (7.1 g, 18%). Analytical HPLC Rₜ=13.75 min; ESI-MS m/z calcd for molecular formula C₂₉H₃₆ClIN₅O₅S₃⁺[M+H]⁺ 792.06, found 792.20.

### Synthesis of Example 49

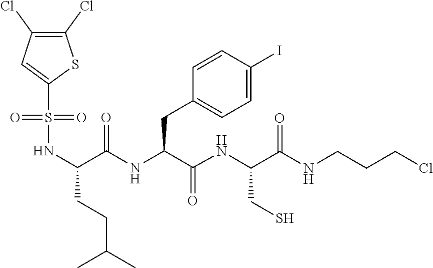 [SMILES: CC(C)CC[C@H](NS(=O)(=O)c1cc(Cl)c(Cl)s1)C(=O)N[C@@H](Cc1ccc(I)cc1)C(=O)N[C@@H](CS)C(=O)NCCCCl]

Example 49 was synthesized according to the method used for the synthesis of Example 30, using the corresponding sulphonyl chloride and 3-chloropropylamine. Crude product was purified by reverse phased-HPLC. After vac-off the volatile and freeze drying the water white fluffy material Example 49 (7.5 g, 18%). Analytical HPLC Rₜ=18.15 min; ESI-MS m/z calcd for molecular formula C₂₆H₃₅Cl₃IN₄O₅S₃⁺[M+H]⁺ 810.99, found 811.2.

### Synthesis of Example 50

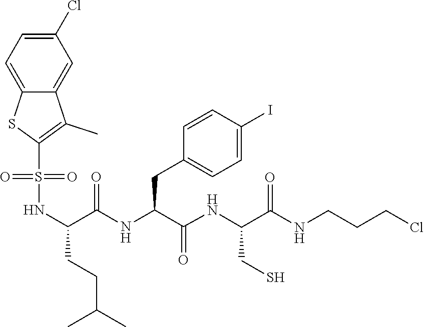 [SMILES: Cc1c(S(=O)(=O)N[C@@H](CCC(C)C)C(=O)N[C@@H](Cc2ccc(I)cc2)C(=O)N[C@@H](CS)C(=O)NCCCCl)sc2ccc(Cl)cc12]

Example 50 was synthesized according to the method used for the synthesis of Example 30, using the corresponding sulphonyl chloride and 3-Chloropropylamine. Crude product was purified by reverse phased-HPLC. After vac-off the volatile and freeze drying the water white fluffy material Example 50 (8 mg, 19%). Analytical HPLC Rₜ=min; ESI-MS m/z calcd for molecular formula C₃₁H₄OCl₂IN₄O₅S₃⁺[M+H]⁺ 841.04, found 841.2.

### Synthesis of Example 51

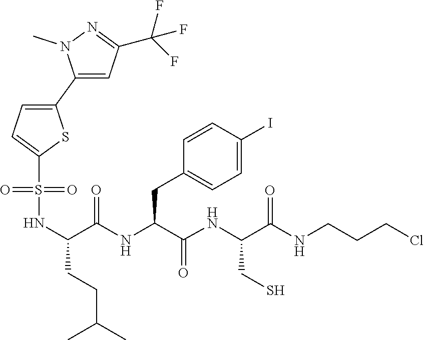 [SMILES: CC(C)CC[C@H](NS(=O)(=O)c1ccc(-c2cc(C(F)(F)F)nn2C)s1)C(=O)N[C@@H](Cc1ccc(I)cc1)C(=O)N[C@@H](CS)C(=O)NCCCCl]

Example 51 was synthesized according to the method used for the synthesis of Example 30, using the corresponding sulphonyl chloride and 3-Chloropropylamine. Crude product was purified by reverse phased-HPLC. After vac-off the volatile and freeze drying the water white fluffy material Example 51 (9 mg, 20%). LCMS Rₜ=2.61 min; ESI-MS m/z calcd for molecular formula C₃₁H₄₀ClF₃IN₆O₅S₃⁺[M+H]⁺ 891.09, found 891.3.

### Synthesis of Example 52

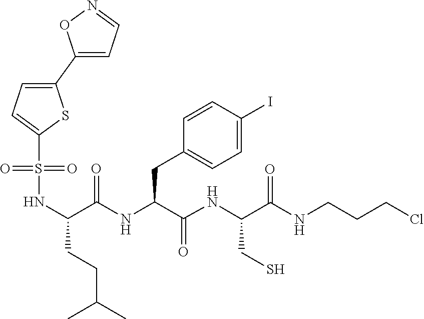 [SMILES: CC(C)CC[C@H](NS(=O)(=O)c1ccc(-c2ccno2)s1)C(=O)N[C@@H](Cc1ccc(I)cc1)C(=O)N[C@@H](CS)C(=O)NCCCCl]

Example 52 was synthesized according to the method used for the synthesis of Example 30, using corresponding sulphonyl chloride and 3-Chloropropylamine. Crude product was purified by reverse phased-HPLC. After vac-off the volatile and freeze drying the water white fluffy material Example 52 (9.5 mg, 23%). LCMS Rₜ=2.40 min; ESI-MS m/z calcd for molecular formula C₂₉H₃₈ClIN₅O₆S₃⁺[M+H]⁺ 810.07, found 810.20.

### Synthesis of Example 53

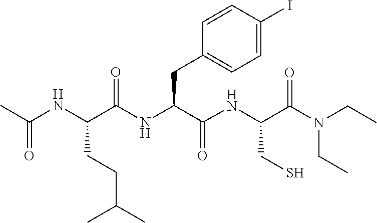 [SMILES: CCN(CC)C(=O)[C@H](CS)NC(=O)[C@H](Cc1ccc(I)cc1)NC(=O)[C@H](CCC(C)C)NC(C)=O]

Example 53 was synthesized according to the method used for the synthesis of Example 30 using corresponding diethylamine. Crude product was purified by reverse phased-HPLC. After vac-off the volatile and freeze drying the water white fluffy material Example 53. LCMS Rₜ=2.20 min; ESI-MS m/z calcd for molecular formula C₂₅H₄₀IN₄O₄S⁺[M+H]⁺ 619.18, found 619.40.

### Synthesis of Example 54

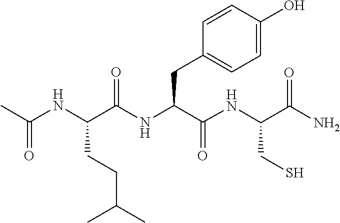 [SMILES: CC(=O)N[C@@H](CCC(C)C)C(=O)N[C@@H](Cc1ccc(O)cc1)C(=O)N[C@@H](CS)C(N)=O]

Example 54 was synthesized according to the method used for the synthesis of Example 1. Crude product was purified by reverse phased-HPLC. After vac-off the volatile and freeze drying the water white fluffy material Example 54. Analytical HPLC Rₜ=8.33 min; ESI-MS m/z calcd for molecular formula C₂₁H₃₂N₅O₄S⁺[M+H]⁺ 453.22, found 453.20.

### Synthesis of Example 55

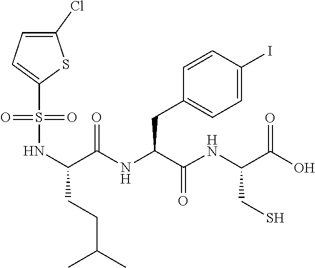 [SMILES: CC(C)CC[C@H](NS(=O)(=O)c1ccc(Cl)s1)C(=O)N[C@@H](Cc1ccc(I)cc1)C(=O)N[C@@H](CS)C(=O)O]

Example 55 was synthesized according to the method used for the synthesis of Example 1. Crude product was purified by reverse phased-HPLC. After vac-off the volatile and freeze drying the water white fluffy material Example 55 (17 mg, 59%). LCMS Rₜ=2.64 min; ESI-MS m/z calcd for molecular formula C₂₃H₃₄ClIN₄O₅S₃⁺[M+H]⁺ 702.06, found 702.30.

### Synthesis of Example 56

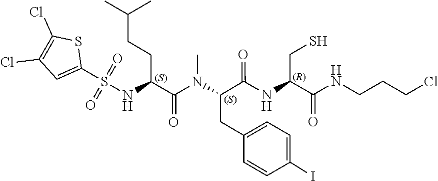

Example 56 was synthesized according to the method used for the synthesis of Example 30.

### Synthesis of Example 57

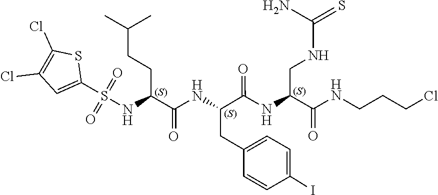

Example 57 was synthesized according to the method used for the synthesis of Example 30.

### Synthesis of Example 58

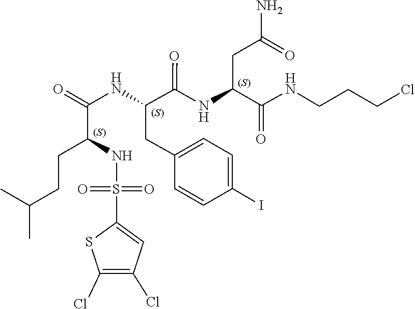

Example 58 was synthesized according to the method used for the synthesis of Example 30.

### Synthesis of Example 59

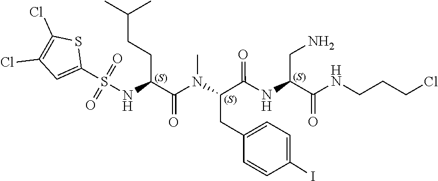

Example 59 was synthesized according to the method used for the synthesis of Example 30.

### Synthesis of Example 60

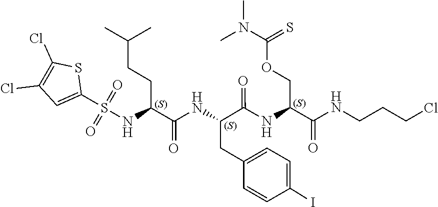

Example 60 was synthesized according to the method used for the synthesis of Example 30.

### Synthesis of Example 61

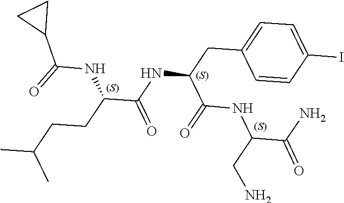

Example 61 was synthesized according to the method used for the synthesis of Example 30.

### Synthesis of Example 62

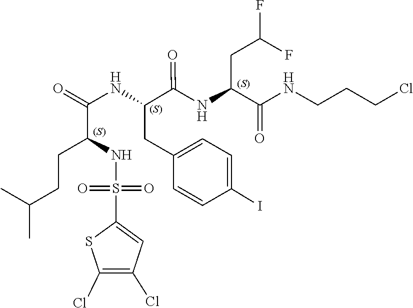

Example 62 was synthesized according to the method used for the synthesis of Example 30.

### Synthesis of Example 63

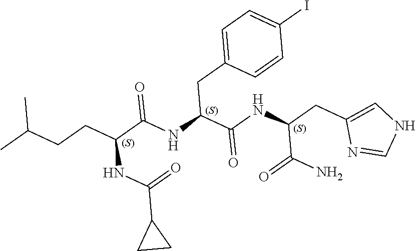

Example 63 was synthesized according to the method used for the synthesis of Example 30.

### Synthesis of Example 64

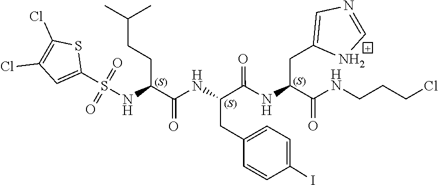

Example 64 was synthesized according to the method used for the synthesis of Example 30.

### Synthesis of Example 65

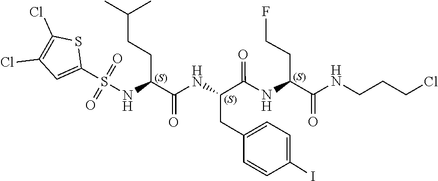

Example 65 was synthesized according to the method used for the synthesis of Example 30.

### Synthesis of Example 66

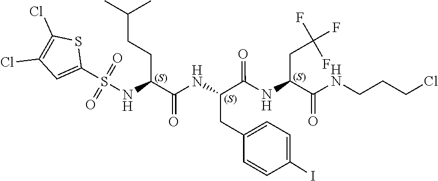

Example 66 was synthesized according to the method used for the synthesis of Example 30.

### Synthesis of Example 67

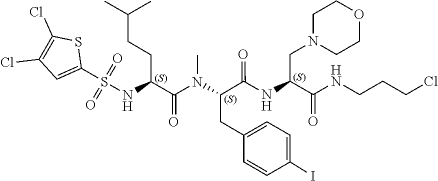

Example 67 was synthesized according to the method used for the synthesis of Example 30.

### Synthesis of Example 68

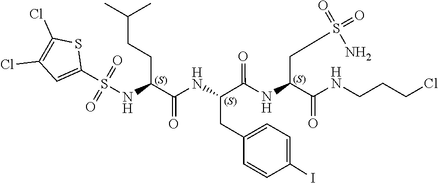

Example 68 was synthesized according to the method used for the synthesis of Example 30.

### Synthesis of Example 69

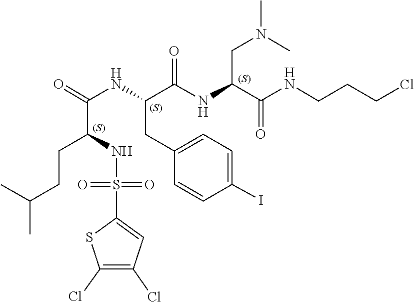

Example 69 was synthesized according to the method used for the synthesis of Example 30.

### Synthesis of Example 70

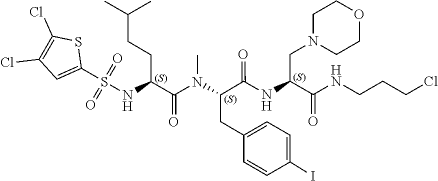

Example 70 was synthesized according to the method used for the synthesis of Example 30.

### Synthesis of Example 71

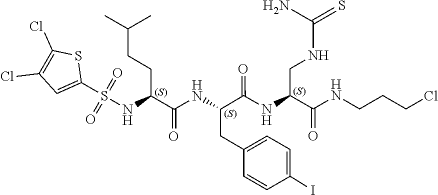

Example 71 was synthesized according to the method used for the synthesis of Example 30.

### Synthesis of Example 72

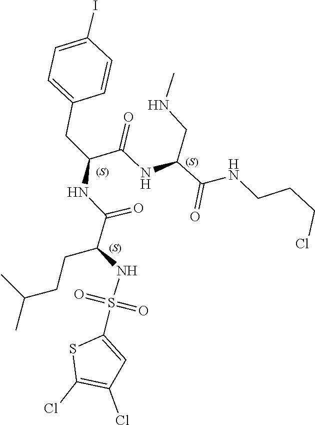

Example 72 was synthesized according to the method HN used for the synthesis of Example 30.

### Synthesis of Example 73

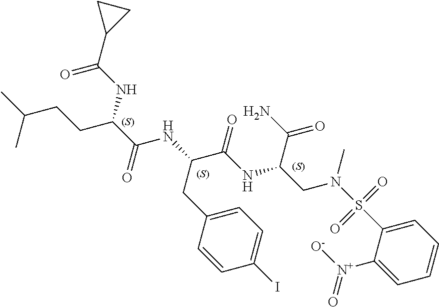

Example 73 was synthesized according to the method used for the synthesis of Example 30.

### Synthesis of Example 74

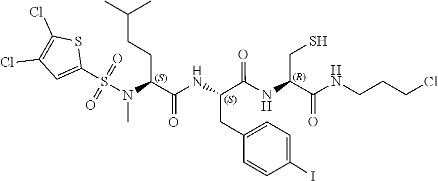

Example 74 was synthesized according to the method used for the synthesis of Example 30.

### Synthesis of Example 75

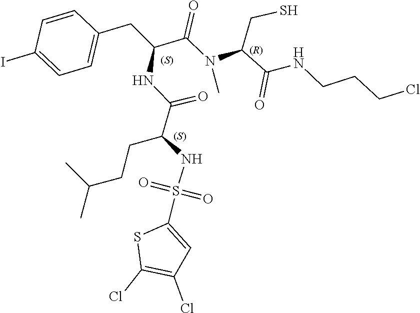

Example 75 was synthesized according to the method used for the synthesis of Example 30.

### Synthesis of Example 76

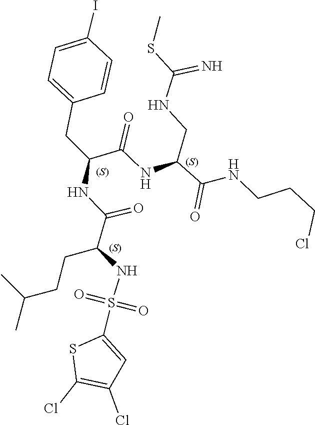

Example 76 was synthesized according to the method used for the synthesis of Example 30.

### Synthesis of Example 77

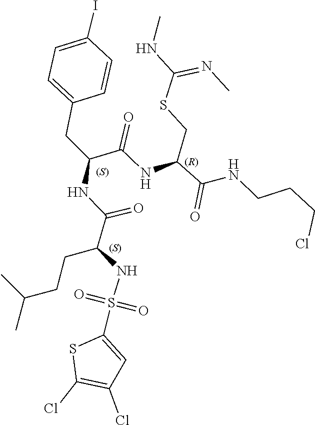

Example 77 was synthesized according to the method used for the synthesis of Example 30.

### Synthesis of Example 78

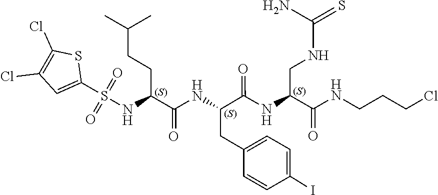

Example 78 was synthesized according to the method used for the synthesis of Example 30.

### Synthesis of Example 79

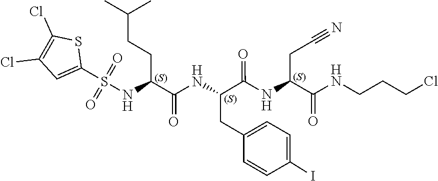

Example 79 was synthesized according to the method used for the synthesis of Example 30.

### Synthesis of Example 80

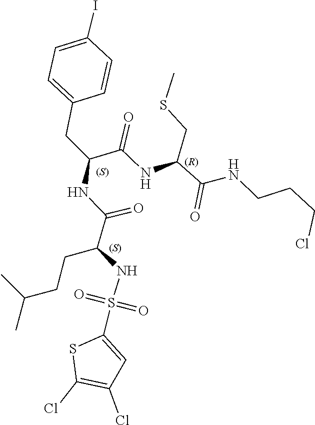

Example 80 was synthesized according to the method used for the synthesis of Example 30.

### Assessment of HIF-1α and 2α Binding Activity in In Vitro Assays

The binding affinity of the tripeptides was determined against the recombinantly expressed PAS-B domain of both HIF-1α and 2α using microscale thermophoresis (MST).

HIF-α proteins were labelled with Monolith NT-647 labelling dye (Nanotemper Technologies GmbH) according to the manufacturer's instructions. MST experiments were performed on a Monolith NT.115 system (Nanotemper Technologies GmbH), in assay buffer containing 50 nM labelled protein, 10% DMSO and 0.05% TWEEN-20. MST measurements were performed using 50% LED and 50% MST power.

### Results

| Example | 1a - MST Kd (uM) |
|---|---|
| 1 | >40 |
| 2 | 8.5 |
| 3 | 70 |
| 4 | 5.4 |
| 5 | >50 |
| 7 | 2.8 |
| 8 | 17 |
| 9 | 78 |
| 10 | 75 |
| 11 | 13 |
| 12 | 7.4 |
| 13 | 5.8 |
| 14 | 22 |
| 15 | 257 |
| 16 | 18 |
| 17 | 9.5 |
| 18 | 3.6 |
| 19 | 8.4 |
| 22 | 34 |
| 23 | 13 |
| 24 | 25 |
| 25 | 54 |
| 27 | 6.1 |
| 28 | 8 |
| 29 | 24 |
| 31 | 22 |
| 32 | 15 |
| 33 | 11 |
| 34 | 16 |
| 35 | 93 |
| 36 | 4.4 |
| 37 | 1.8 |
| 39 | 17 |
| 40 | 100 |
| 41 | >100 |
| 42 | 25 |
| 44 | 15 |
| 45 | 12 |
| 46 | 27 |
| 47 | 12 |
| 48 | 26 |
| 49 | 8.9 |
| 50 | 6.7 |
| 51 | 4.9 |
| 52 | 14 |
| 53 | 59 |
| 54 | 128 |
| 55 | 8.4 |
| 56 | 2.1 |
| 58 | >500 |
| 59 | >500 |
| 63 | 46.3 |
| 66 | >125 |
| 67 | >500 |
| 68 | >500 |
| 72 | >500 |
| 74 | 4.8 |
| 80 | 19 |

| Example | 2a - MST Kd (uM) |
|---|---|
| 17 | 5.2 |
| 42 | 11.7 |
| 48 | 2.6 |
| 51 | 3.1 |

### CONCLUSIONS

The MST assays show that the tested compounds have binding activity for the 1α and 2α PAS-B domains. Therefore, these compounds are expected to be useful in the treatment of associated diseases and conditions.

### Assessment of HIF Inhibition

Protocol for MCF7 qPCR Assay

Compounds of Example Nos. 2, 13, 27, 28, 49 and 55 were individually added to MCF7 cells at a final concentration of 25 uM in 1% final DMSO. Following treatment, cells were incubated in normoxia for 4 h, then in hypoxia for 16 h. RNA was extracted from treated and control cells and subsequently reverse-transcribed into cDNA. Gene expression levels of 18S and CAIX genes were measured by qPCR. For each sample, expression levels of CAIX were normalised to levels of internal reference gene, 18S.

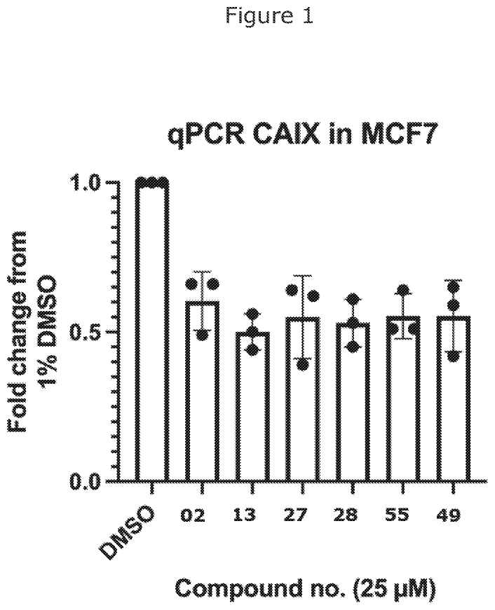

Results are expressed as fold-change compared to hypoxic control cells (treated with 1% DMSO). The results are shown in FIG. 1.

These results indicate that HIF activity is being inhibited in the cells treated with compounds of the disclosure.

1-34. (canceled)

35) A compound of formula (1): 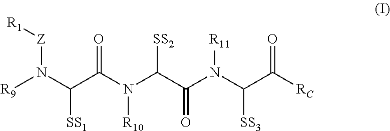
wherein Z═CO or SO₂;
R₉, R₁₀ and R₁₁ are independently H or methyl;
R₁=straight-chain or branched C₁-C₈ alkyl; CH-(3-6 membered ring)₂; (CH₂)₁-₃O(CH₂)₀₋₃CH₃; or (CH₂)₀₋₃R₂;
R₂═NH(CH₂)₀₋₃CH₃; a 8-12 membered bicyclic ring optionally substituted by one or more of C₁-C₃ alkyl, HaI, CHHaI₂, CH₂HaI, and CHaI₃; or a 3-6 membered ring optionally substituted by one or more of C₁-C₃ alkyl, HaI, CHHaI₂, CH₂HaI, CHaI₃, CO(CH₂)₀₋₂CH₃, nitro group and (O)₀₋₁-3-6 membered ring optionally substituted by one or more of C₁-C₃ alkyl, HaI, CHHaI₂, CH₂HaI, and CHaI₃;
R_{C} is (i), (ii) or (iii):
wherein (i) is: 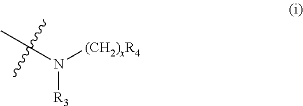
wherein R₃═H; or straight-chain or branched C₁-C₈ alkyl;
X=an integer of from 0 to 8;
R₄═H; CH₃; CHHaI₂; CH₂HaI; CHaI₃; HaI; CCH; NR₅R₆; CH(COOMe)(CH₂)₁₋₄NHC(NH₂)(NH); or 3-6 membered ring optionally substituted by one or more of C₁-C₃ alkyl, HaI, NR₇R₈, and COCH₃;
R₅═H; or straight-chain or branched C₁-C₃ alkyl;
R₆═H; or straight-chain or branched C₁-C₃ alkyl; or 3-6 membered ring;
R₇═H; or straight-chain or branched C₁-C₃ alkyl;
R₈═H; or straight-chain or branched C₁-C₃ alkyl;
wherein (ii) is a 5-6 membered heterocycle comprising at least one nitrogen atom and optionally substituted by one or more of C₁-C₃ alkyl, HaI, CHHaI₂, CH₂HaI, CHaI₃, NR₇R₈, and COCH₃, wherein R_{c} is bonded via the at least one nitrogen atom;
wherein (iii) is OH;
wherein HaI is a halogen;
wherein SS₁, SS₂ and SS₃ are independently selected from one of the side-chains in list (a), list (b) or list (c): 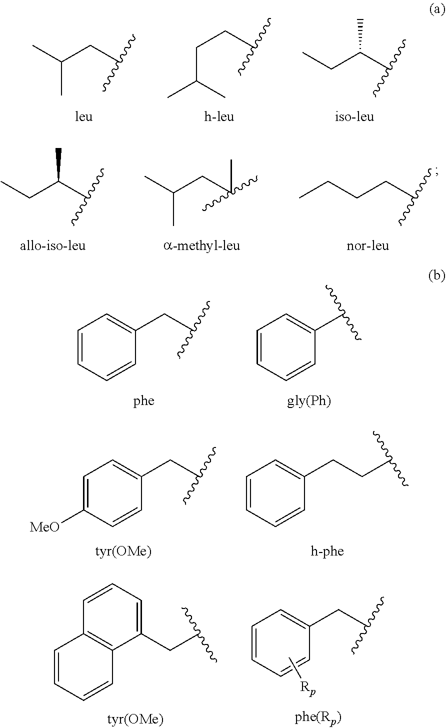
wherein the phe(R_{P}) ring is substituted with one, two, or three R_{P} groups; and each R_{P} is independently I, Br, Cl, F, OH, Bz, NO₂, CN, or CF₃; 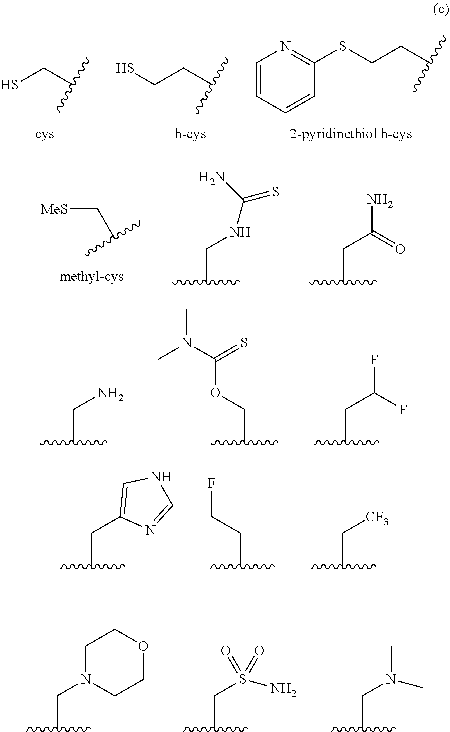 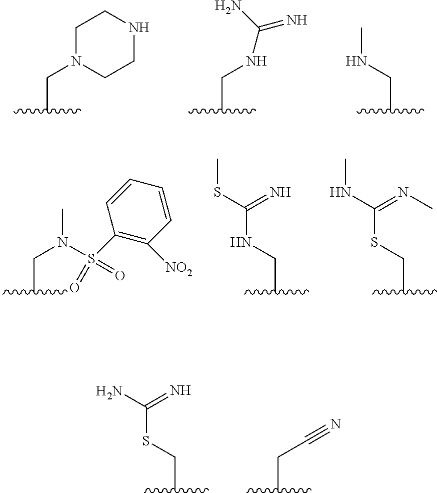
wherein SS₁, SS₂ and SS₃ are each selected from different lists;
or a pharmaceutically acceptable salt thereof.

36) The compound of claim 35, wherein R₉, R₁₀ and/or R₁₁ are H.

37) The compound of claim 35, wherein list (a) is:
(a) leu, h-leu, iso-leu, allo-iso-leu, and nor-leu.

38) The compound of claim 35, wherein list (b) is:
(b) phe, gly(Ph), tyr(OMe), h-phe, and phe(R_{P});

39) The compound of claim 35, wherein list (c) is:
(c) cys, h-cys, 2-pyridinethiol h-cys, and methyl-cys.

40) The compound of claim 35, wherein R₉, and/or R₁₀ are methyl.

41) The compound of claim 35, wherein Z represents SO₂.

42) The compound of claim 35, wherein R₁ represents a straight-chain or branched C₁-C₈ alkyl, CH-(3-6 membered ring)₂ or (CH₂)₀₋₂R₂.

43) The compound of claim 35, wherein R₂ represents NH(CH₂)₀₋₁CH₃; an 8-10 membered bicyclic ring optionally substituted by one or more of C₁-C₃ alkyl and HaI; or a 3-6 membered ring optionally substituted by one or more of C₁-C₃ alkyl, HaI and (O)₀₋₁-3-6 membered ring optionally substituted by one or more of C₁-C₃ alkyl, HaI and CHaI₃.

44) The compound of claim 35, wherein R₂ represents:
a) an 8-9 membered bicyclic ring, optionally substituted by one or more of C₁-C₃ alkyl and HaI; or
b) a 3-6 membered ring, optionally substituted by one or more of C₁-C₃ alkyl, HaI and (O)₀₋₁-3-6 membered ring optionally substituted by one or more of C₁-C₃ alkyl, HaI and CHaI₃; or
c) NH(CH₂)₀CH₃, phenyl, furan, morpholine, cyclopropane, diphenyl ether, thiophene, 2-chlorothiophene, 1-methylimidazole, 2-bromothiophene, 2,3-dichlorothiophene, 2-chloro-3-nitrothiophene, 5-Chloro-3-methyl-1-benzothiophene, 5-(2-Thienyl)-1,2-oxazole or 1-methyl-5-thien-2-yl-3-(trifluoromethyl)-1H-pyrazole, 2-chlorothiophene, 1-methyl-5-thien-2-yl-3-(trifluoromethyl)-1H-pyrazole, 5-Chloro-3-methyl-1-benzothiophene, 2,3-dichlorothiophene, thiophene, diphenyl ether, or phenyl.

45) The compound of claim 35, wherein R_{c} is formula (i) or (iii).

46) The compound of claim 35, wherein:
a) R₃ represents H or a straight-chain or branched C₁-C₄ alkyl; and/or
b) X is an integer of from 0 to 6.

47) The compound of claim 35, wherein:
a) R₄ represents H; CHaI₃; HaI; CCH; NR₅R₆; CH(COOMe)(CH₂)₁₋₄(NH)C(NH₂)(NH); or 3-6 membered ring optionally substituted by one or more of C₁-C₃ alkyl, HaI, NR₇R₈ and COCH₃; HaI; or 3-6 membered ring optionally substituted by one or more of C₁-C₃ alkyl, HaI, NR₇R₈, and COCH₃; and/or
b) (CH₂)_{X}R₄ represents H, (CH₂)₁CH₃, (CH₂)₂CH₃, (CH₂)₁CF₃, (CH₂)₀-cyclopropane, (CH₂)₁CCH, (CH₂)₃C₁, (CH₂)₂NHPh, (CH₂)₅NH₂, (CH₂)₆NH₂, CH(COOMe)(CH₂)₃N(H)C(NH₂)(NH), (CH₂)₀-tetrahydropyran-4-yl, (CH₂)₁Ph, (CH₂)₁-furan-2-yl, (CH₂)₁-thiophen-2-yl or (CH₂)₁-pyridin-2-yl, (CH₂)₃Cl, (CH₂)₂CH₃, (CH₂)₁Ph or (CH₂)₁-thiophen-2-yl.

48) The compound of claim 35, wherein each R_{P} independently represents I, Br, Cl, F, or OH.

49) The compound of claim 35, wherein SS₁ is selected from (a), SS₂ is selected from (b) and SS₃ is selected from (c).

50) A method of preventing or reducing hypoxia induced expression from a promoter that comprises one or more hypoxia-responsive elements under hypoxic conditions, by administering the compound of claim 35.

51) A method of treatment or prevention of:
a) a disease, disorder or condition that experiences a hypoxic environment and requires the typical hypoxia response for maintenance, and/or;
b) any other disease treatable or preventable by inhibition of dimerization of HIF-1a with HIF1-b and HIF2a with HIF1b and/or inhibits the activity of HIF-1 and HIF-2 and/or HIF-1 or HIF-2 signalling, and/or;
c) a disease, disorder or condition in which it is desirable to repress hypoxia induced gene expression;
by administering the compound of claim 35.

52) The method of claim 51 wherein the disease, disorder or condition is selected from Von Hippel-Lindau disease, tumours and cancer.

53) A compound of claim 35, wherein the compound is of formula (IV): 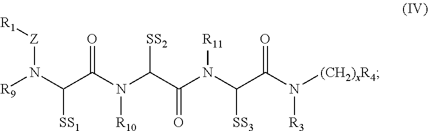
wherein Z, R₁, R₂, R₃, X, R₄, R₉, R₁₀, R₁₁, SS₁, SS₂ and SS₃ are as defined in claim 35;
wherein one or more of SS₁, SS₂ and SS₃ comprises a nucleophilic functional group and has a protecting group, on said functional group;
or a protected derivative thereof or salt thereof.

54) A compound of claim 35, wherein the compound is of formula (V): 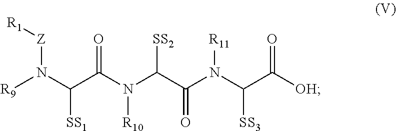
wherein Z, R₁, R₂, R₉, R₁₀, R₁₁, SS₁, SS₂ and SS₃ are as defined in claim 35;
or a protected derivative thereof or salt thereof.
